(19) United States
(12) Patent Application Publication
(10) Pub. No.: US 2026/0158187 A1
(43) Pub. Date: Jun. 11, 2026

(54) OCULAR SEALANT FORMULATIONS AND SYSTEMS AND METHODS OF PREPARATION AND USE THEREOF

(71) Applicant: OCULAR THERAPEUTIX, INC., Bedford, MA (US)

(72) Inventors: Nelson BELLO, Revere, MA (US); Dinesh HASWANI, Acton, MA (US); Rami EL-HAYEK, Norwood, MA (US); Peter JARRETT, Burlington, MA (US)

(21) Appl. No.: 18/706,998

(22) PCT Filed: Nov. 12, 2021
(86) PCT No.: PCT/US2021/059128

§ 371 Date: May 2, 2024

(51) Int. Cl. A61L 24/04; C08G 65/333; C08J 3/24

(52) U.S. Cl. CPC A61L 24/046; C08G 65/33303; C08G 65/33396; C08J 3/246; C08J 3/247; C08J 2371/02

(57) ABSTRACT

Disclosed are ocular sealant formulations and systems for treating ocular wounds. The ocular sealant formulations are administered to a subject by applying the sealant to a surface of the subject's eye. Also disclosed are methods of preparing ocular sealant formulations and systems and methods of using such formulations and systems.

### TECHNICAL FIELD

The present invention relates to ocular sealant formulations and in certain embodiments to systems for preparing ocular sealant formulations. Also disclosed in one or more embodiments are methods of preparing such systems and ocular sealant formulations and methods of using such systems and ocular sealant formulations.

### BACKGROUND

Wounds on the surface of the eye may be caused by trauma (e.g., scratches, cuts, etc.) or surgery (e.g., incisions, abrasions, etc.). Such wounds often can be painful and the brushing of the eyelid against the eye can aggravate the pain. Several techniques exist to treat ocular wounds, including bandage therapeutic lenses, non-steroidal anti-inflammatoires, steroids, antibiotics and analgesics. A bandage therapeutic lens, for example, may slide over the wound when positioned on the eye. In at least some instances, however, this positioning may decrease the therapeutic benefit when the lens slides along the delicate underlying tissue, for example when a patient blinks. A need, therefore, remains for alternative methods and compositions to bandage ocular wounds. In view of the drawbacks and challenges experienced with current available treatments, novel treatment methods that effectively deliver glucocorticoids, that are effective over a period of one or more weeks and avoid the need for daily glucocorticoid administrations, would be beneficial to patients.

### OBJECTS AND SUMMARY OF THE INVENTION

It is an object of certain embodiments of the present invention to provide an ocular sealant system, comprising a nucleophilic group-containing crosslinker component comprising less than about 500 ppm by weight water, an electrophilic group-containing multi-arm-polymer precursor component comprising less than about 1 wt. % water, and a solvent comprising at least one salt.

It is an object of certain embodiments of the present invention to provide an ocular sealant system, comprising a nucleophilic group-containing crosslinker component comprising less than about 500 ppm by weight water, an electrophilic group-containing multi-arm-polymer precursor component comprising less than about 1 wt. % water, a solvent comprising at least one salt, and an applicator comprising a thin film of the nucleophilic group-containing crosslinker component on a surface of the applicator.

It is an object of certain embodiments of the present invention to provide a method of preparing an ocular sealant formulation system, comprising depositing a nucleophilic group-containing crosslinker component on a surface of the container, drying the nucleophilic group-containing crosslinker component and the container, depositing a molten electrophilic group-containing multi-arm-polymer precursor component on a surface of the container, and sealing the container having the nucleophilic group-containing crosslinker component and the electrophilic group-containing multi-arm-polymer precursor component thereon in a storage container.

It is an object of certain embodiments of the present invention to provide an ocular sealant dosage form, comprising a compressed mixture of: a nucleophilic group-containing crosslinker component; an electrophilic group-containing multi-arm-polymer precursor component; and at least one excipient, wherein the ocular sealant dosage form comprises less than about 5 wt. % water.

It is an object of certain embodiments of the present invention to provide an ocular sealant system, comprising an ocular sealant dosage form, comprising: an electrophilic group-containing multi-arm-polymer precursor component; a polyethylene glycol (PEG) amine component; and at least one excipient, wherein the ocular sealant dosage form comprises less than about 5 wt. % water, and a solvent comprising at least one salt.

It is an object of certain embodiments of the present invention to provide method of preparing an ocular sealant dosage form, comprising forming particles comprising an electrophilic group-containing multi-arm-polymer precursor component, a nucleophilic group-containing crosslinker component and at least one excipient, combining the particles to form a combination, and compressing the combination to form at least one tablet.

It is an object of certain embodiments of the present invention to provide an ocular sealant formulation, comprising a polymer network formed by combining: a nucleophilic group-containing crosslinker component comprising less than about 500 ppm by weight water, an electrophilic group-containing multi-arm-polymer precursor component comprising less than about 1 wt. % water, and a solvent comprising at least one salt.

One or more of these objects of the present invention and others are solved by one or more embodiments of the invention as disclosed and claimed herein.

The individual aspects of the present invention are disclosed in the specification and claimed in the independent claims, while the dependent claims recite particular embodiments and variations of these aspects of the invention. Details of the various aspects of the present invention are provided in the detailed description below.

### BRIEF DESCRIPTION OF THE DRAWINGS

FIG. 1 shows a perspective view of an ocular sealant formulation system according to certain embodiments disclosed herein.

FIG. 2A shows a top schematic view of a container (e.g., a tray) of an ocular sealant formulation system according to certain embodiments disclosed herein.

FIG. 2B shows a side view of a container (e.g., a tray) of an ocular sealant formulation system according to certain embodiments disclosed herein.

FIG. 2C shows a perspective view of an applicator of an ocular sealant formulation system according applying occular sealant formulation to any eye according to certain embodiments disclosed herein.

FIG. 3A shows a scanning electron microscopy (SEM) image of a lyophilized polyethylene glycol solid in accordance with the prior art.

FIG. 3B shows a SEM image of a solidified printed melt of polyethylene glycol according to certain embodiments disclosed herein.

FIG. 4A shows a perspective view of an ocular sealant formulation system according to certain embodiments disclosed herein.

FIG. 4B shows an applicator of an ocular sealant formulation system having a nucleophilic group-containing crosslinker component deposited thereon according to certain embodiments disclosed herein.

FIG. 4C shows an applicator of an ocular sealant formulation system having a thin film of a nucleophilic group-containing crosslinker component deposited thereon according to certain embodiments disclosed herein.

FIG. 5 shows an ocular sealant formulation system according to certain embodiments disclosed herein.

FIG. 6A shows an embodiment of a tray for an ocular sealant formulation system accordong to certain embodiments disclosed herein.

FIG. 6B shows an embodiment of a tray and an applicator for an ocular sealant formulation system accordong to certain embodiments disclosed herein.

FIG. 7 shows a flow chart of a method of preparing an ocular sealant dosage form according to certain embodiments disclosed herein.

FIG. 8 is a bar graph comparing the water content of a baked nucleophilic group-containing crosslinker component to that of a comparative lyophilized nucleophilic group-containing crosslinker component.

FIG. 9 is a chart showing the N-hydroxysuccinimidyl (NHS) percent substitution of a printed electrophilic group-containing multi-arm-polymer precursor component as a function of time when heating at about 85° C.

### DEFINITIONS

The term “ocular” as used herein refers to the eye in general, or any part or portion of the eye (as an “ocular sealant” according to the invention refers to an a formulation that can in principle be administered to the surface of the eye). The present invention in certain embodiments is directed to topical administration of an ocular sealant formulation, and to the treatment of allergic conjunctivitis, as further disclosed herein.

The term “biodegradable” as used herein refers to a material or object, which becomes degraded in vivo, i.e., within a human or animal body.

A “hydrogel” is a three-dimensional network of one or more hydrophilic natural or synthetic polymers (as disclosed herein) that can swell in water and hold an amount of water while maintaining or substantially maintaining its structure, e.g., due to chemical or physical cross-linking of individual polymer chains. Due to their high water content, hydrogels are soft and flexible, which makes them very similar to natural tissue. In the present invention the term “hydrogel” is used to refer both to a hydrogel in the hydrated state when it contains water (e.g. after the hydrogel has been formed in an aqueous solution, or after the hydrogel has been hydrated or (re-)hydrated once administered on the eye or otherwise immersed into an aqueous environment) and to a hydrogel in its/a dry (dried/dehydrated) state when it has been dried to a low water content of e.g. not more than 1% by weight. In the present invention, wherein an active principle is contained (e.g. dispersed) in a hydrogel, the hydrogel may also be referred to as a “matrix”.

The term “polymer network” as used herein describes a structure formed of polymer chains (of the same or different molecular structure and of the same or different average molecular weight) that are cross-linked with each other. Types of polymers suitable for the purposes of the present invention are disclosed herein. The polymer network may be formed with the aid of a crosslinking agent as also disclosed herein.

The term “amorphous” refers to a polymer or polymer network which does not exhibit crystalline structures in X-ray or electron scattering experiments.

The term “semi-crystalline” refers to a polymer or polymer network which possesses some crystalline character, i.e., exhibits some crystalline properties in X-ray or electron scattering experiments.

The term “precursor” or “polymer precursor” herein refers to those molecules or compounds that are reacted with each other and that are thus connected via crosslinks to form a polymer network and thus the hydrogel matrix. While other materials might be present in the hydrogel, such as active agents, visualization agents or buffers (e.g., a salt), they are not referred to as “precursors”.

The molecular weight of a polymer precursor as used for the purposes of the present invention and as disclosed herein may be determined by analytical methods known in the art. The molecular weight of polyethylene glycol can for example be determined by any method known in the art, including gel electrophoresis such as SDS-PAGE (sodium dodecyl sulphate-polyacrylamide gel electrophoresis), gel permeation chromatography (GPC), including GPC with dynamic light scattering (DLS), liquid chromatography (LC), as well as mass spectrometry such as matrix-assisted laser desorption/ionization-time of flight (MALDI-TOF) spectrometry or electrospray ionization (ESI) mass spectrometry. The molecular weight of a polymer, including a polyethylene glycol precursor as disclosed herein, is an average molecular weight (based on the polymer's molecular weight distribution), and may therefore be indicated by means of various average values, including the weight average molecular weight (Mw) and the number average molecular weight (Mn). Any of such average values may be used in the context of the present invention. In certain embodiments, the average molecular weight of the polyethylene glycol units or other precursors as disclosed herein is the number average molecular weight.

The parts of the precursor molecules that are still present in a final polymer network are also called “units” herein. The “units” are thus the building blocks or constituents of a polymer network forming the hydrogel. For example, a polymer network suitable for use in the present invention may contain identical or different polyethylene glycol units as further disclosed herein.

As used herein, the term “crosslinking agent” or “crosslinker” or “crosslinking component” refers to any molecule that is suitable for connecting precursors via crosslinks to form the polymer network and thus the hydrogel matrix. In certain embodiments, crosslinking agents may be low-molecular weight compounds or may be polymeric compounds as disclosed herein.

As used herein, the term “ocular surface” comprises the conjunctiva and/or the cornea, together with elements such as the lacrimal apparatus, including the lacrimal punctum, as well as the lacrimal canaliculus and associated eyelid structures. Within the meaning of this invention, the ocular surface encompasses also the aqueous humor.

As used herein, the terms “tear fluid” or “tears” or “tear film” refer to the liquid secreted by the lacrimal glands, which lubricates the eyes. Tears are made up of water, electrolytes, proteins, lipids, and mucins.

As used herein, the terms “sealant” or “ocular sealant” or “seal” refer to a substance that is applied to the incisions or wound on an ocular surface for the purpose of sealing/closing the wound.

As used herein, the terms “administration” or “administering” or “administered” etc. in the context of the ocular sealant formulation of the present invention refer to the process of combining the individual components of the ocular sealant formulation and applying the formulation to a surface of an eye. Thus, “administering an ocular sealant formulation” or similar terms refer to mixing and/or applying a sealant to a surface of an eye. The terms “applying” or “brushing” or “spreading” etc. in the context of the ocular sealant formulations of the present invention refer to topical application of these products onto the eye.

The terms “API”, “active (pharmaceutical) ingredient”, “active (pharmaceutical) agent”, “active (pharmaceutical) principle”, “(active) therapeutic agent”, “active”, and “drug” are used interchangeably herein and refer to the substance used in a finished pharmaceutical product (FPP) as well as the substance used in the preparation of such a finished pharmaceutical product, intended to furnish pharmacological activity or to otherwise have direct effect in the diagnosis, cure, mitigation, treatment or prevention of a disease, or to have direct effect in restoring, correcting or modifying physiological functions in a patient.

As used herein, the term “therapeutically effective” refers to the amount of drug or active agent (i.e. glucocorticoid) required to produce a desired therapeutic response or result after administration. For example, in the context of the present invention, one desired therapeutic result would be the reduction of symptoms associated with allergic conjunctivitis such as ocular itching and conjunctival redness.

The term “average” as used herein refers to a central or typical value in a set of data, which is calculated by dividing the sum of the values in the set by their number.

As used herein, the term “about” in connection with a measured quantity refers to the normal variations in that measured quantity, as expected by one of ordinary skill in the art in making the measurement and exercising a level of care commensurate with the objective of measurement and the precision of the measuring equipment.

As used herein, the term “at least about” in connection with a measured quantity refers to the normal variations in the measured quantity, as expected by one of ordinary skill in the art in making the measurement and exercising a level of care commensurate with the objective of measurement and precisions of the measuring equipment and any quantities higher than that. The term “at least about” in connection with a measured quantity refers to the normal variations in the measured quantity, as expected by one of ordinary skill in the art in making the measurement and exercising a level of care commensurate with the objective of measurement and precisions of the measuring equipment and any quantities higher than that. In certain embodiments, the term “at least about” includes the recited number minus 10% and any quantity that is higher such that “at least about 10” would include 9 and anything greater than 9. This term can also be expressed as “about 10 or more.” Similarly, the term “less than about” typically includes the recited number plus 10% and any quantity that is lower such that “less than about 10” would include 11 and anything less than 11. This term can also be expressed as “about 10 or less.”

The term “and/or” as used in a phrase such as “A and/or B” herein is intended to include both “A and B” and “A or B”.

Open terms such as “include,” “including,” “contain,” “containing” and the like as used herein mean “comprising” and are intended to refer to open-ended lists or enumerations of elements, method steps, or the like and are thus not intended to be limited to the recited elements, method steps or the like but are intended to also include additional, unrecited elements, method steps or the like.

The term “up to” when used herein together with a certain value or number is meant to include the respective value or number. For example, the term “up to 25 days” means “up to and including 25 days”.

As used herein, the singular forms “a,” “an”, and “the” include plural references unless the context clearly indicates otherwise. Thus, for example, reference to “a precursor” includes a single precursor as well as to a mixture of two or more precursors; and reference to a “reactant” includes a single reactant as well as a mixture of two or more reactants, and the like.

Reference throughout this specification to “one embodiment” or “an embodiment” means that a particular feature, structure, or characteristic described in connection with the embodiment is included in at least one embodiment. Thus, the appearances of the phrase “in one embodiment” or “in an embodiment” in various places throughout this specification are not necessarily all referring to the same embodiment. In addition, the term “or” is intended to mean an inclusive “or” rather than an exclusive “or.” When the term “about” or “approximately” is used herein, this is intended to mean that the nominal value presented is precise within +10%, such that “about 10” would include from 9 to 11.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

Although the operations of the methods herein are shown and described in a particular order, the order of the operations of each method may be altered so that certain operations may be performed in an inverse order or so that certain operation may be performed, at least in part, concurrently with other operations. In another embodiment, instructions or sub-operations of distinct operations may be in an intermittent and/or alternating manner.

All references disclosed herein are hereby incorporated by reference in their entireties for all purposes (with the instant specification prevailing in case of conflict).

### DETAILED DESCRIPTION

Ocular scratches, cuts, incisions, abrasions, etc. are a common sources of pain and discomfort in subjects (e.g., patients). Current topical drop therapies have limitations including potential for noncompliance, and contact irritation. Although bandage therapeutic lenses may be effective in treating such wounds, in at least some instances, however, positioning the lens on the eye may decrease the therapeutic benefit when the lens slides along the delicate underlying tissue, for example when a patient blinks. A need, therefore, remains for alternative methods and compositions to bandage ocular wounds.

Disclosed herein are ocular sealant formulations and systems containing components for preparing such formulations. For example, provided are ocular sealant formulations formed from a cross-linked polymer network having linkages formed from an electrophilic group-containing multi-arm-polymer precursor component (e.g., polyethylene glycol (PEG)) and a nucleophilic group-containing crosslinker comprising less than about 1 wt. % water component (e.g., trilysine acetate, amine-terminated polyethylene glycol, etc.) comprising less than about 500 ppm by weight water and a solvent comprising at least one salt.

According to one or more embodiments, also disclosed herein are methods of treating a wound on the surface of the eye of a subject in need thereof. The methods include administering to the subject, a therapeutically effective amount of a resorbable ocular sealant fluid. The fluid includes a cross-linked polymer network having, e.g., amide linkages formed from trilysine or amine terminated polyethylene glyocol and an eight (8) ARM 15,000 Da polyethylene glycol (PEG)-Succinimidyl Succinate (hereinafter referred to as “8a15kSS”).

### I. Ocular Sealant Systems

In certain embodiments, the ocular sealant systems of the present invention include a nucleophilic group-containing crosslinker component comprising less than about 500 ppm by weight, less than about 400 ppm by weight, less than about 300 ppm by weight, less than about 250 ppm by weight, less than about 200 ppm by weight, less than about 100 ppm by weight, less than about 50 ppm by weight, less than abou 20 ppm by weight water, or any individual amount or sub-range within these ranges, an electrophilic group-containing multi-arm-polymer precursor component comprising less than about 1 wt. %, less than about 0.5 wt. %, less than about 0.1 wt. %, less than about 0.05 wt. %, or less than about 0.01 wt. % water, or any individual amount or sub-range within these ranges, and a solvent comprising at least one salt. The water content of the nucleophilic component and/or the electrophilic component may be measured using a Karl Fischer analysis. In some embodiments, the KARL FISCHER® analysis may be as set forth in ASTM D6869/ISO 15512. In some embodiments, the KARL FISCHER analysis used to measure the water content may be a melting cure generation method (or loss on drying method) that includes ramping the temperature of a KARL FISCHER oven from 50° C. to 220° C. where spikes in water (μg water detected) and drift (μg/min water) indicate melting and the resulting data can be used to generate melting curves. This KARL FISCHER method was developed to monitor water loss/gain following conditioning of the plastic. The systems may further include an applicator and/or a mixing element configured to combine the nucleophilic group-containing crosslinker component, the electrophilic group-containing multi-arm-polymer precursor component and the solvent in a container (e.g., a tray).

In certain embodiments, the applicator and/or mixing element may include a thin film of the nucleophilic group-containing crosslinker component. The container (e.g., a tray) may include a recess containing a printed melt of an electrophilic group-containing multi-arm-polymer precursor component. The solvent can be added to the recesses and the applicator and/or mixing element having the thin film of the nucleophilic group-containing crosslinker component can be contacted with the solvent and stirred together with the other components.

In certain embodiments, ocular sealant systems may comprise an ocular sealant dosage form, comprising an electrophilic group-containing multi-arm-polymer precursor component, a nucleophilic group-containing crosslinker component, a solubility enhancer, and at least one excipient. Such ocular sealant dosage forms may comprise less than about 95 wt. %, less than about 80 wt. %, less than about 75 wt. %, less than about 50 wt. %, less than about 25 wt. %, less than about 10 wt. %, less than about 5 wt. %, less than about 2.0 wt. %, or any individual amount or sub-range within the ranges, of water. The ocular sealant systems as described herein further include a solvent comprising at least one salt. Such systems may further include an applicator and/or a mixing element configured to combine the dosage form and the solvent in a container (e.g., a tray).

According to various embodiments, the components of the ocular sealant systems as described herein and/or the systems as a whole may have a shelf-life of about 9 months to about 4 years, about 12 months to about 3 years, about 18 months to about 2 years, or any individual duration or sub-range within these ranges. In certain embodiments, ocular sealant systems as described herein may further include a desiccant and/or an oxygen absorber. The desiccant and/or oxygen absorber may be packaged with the container having nucleophilic component and/or the electrophilic component deposited thereon. Additionally or alternatively, the desiccant and/or oxygen absorber may be in the outer container packaging. The desiccant and/or oxygen absorber may be comprised in one or more of a liner of a pouch, a sachet, a canister or a capsule. Suitable desiccants include, but are not limited to, silica gel, a molecular sieve, aluminium oxide, carbon, activated carbon, calcium oxide, calcium sulfate, montmorillonite clay, etc. Suitable oxygen absorbers include, but are not limited to iron or iron oxide.

In some embodiments, the ocular sealant systems as described herein may, as a whole, contain less water than known ocular sealant systems. Ocular sealant systems as described herein may contain less than about 2000 ppm by weight, less than about 1000 ppm by weight, less than about 750 ppm by weight, or any individual amount or sub-range within these ranges, of water.

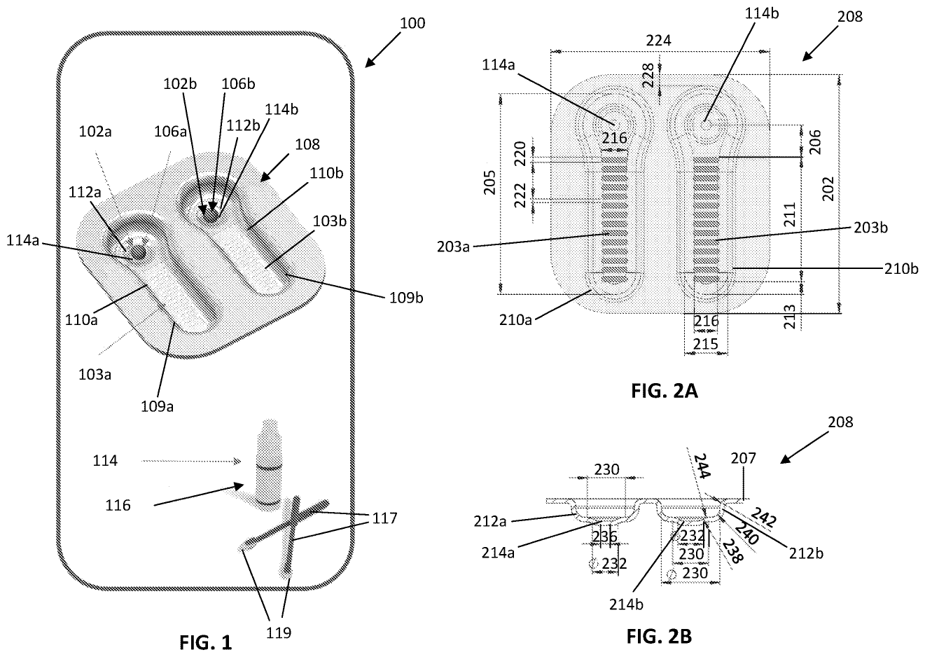

An embodiment of an ocular sealant system 100 is shown in FIG. 1. Ocular sealant system 100 may include a container 108 (e.g., a tray) having one or more recesses 110a, 110b. Container 108 is configured to store both the nucleophilic group-containing crosslinker component and the electrophilic group-containing multi-arm-polymer precursor component. In certain embodiments, the container 108 is formed of a high-density polyethylene, a polycarbonate, a polystyrene, a nylon, an aluminum foil, or combinations thereof. In certain embodiments, the container 108 comprises less than about 0.50 wt. % water, less than about 0.25 wt. % water, less than about 0.20 wt. % water, or less than about 0.15 wt. % water based on the total weight of the container 108 as measured by the Karl Fischer Moisture Analysis (i.e., a Karl Fischer Titration, ASTM D6869/ISO 15512). In some embodiments, the container 108 includes a water absorbance value of less than about 0.50% water, less than about 0.25% water, less than about 0.20% water, or less than about 0.15% water. Reducing and/or eliminating water from the system may increase the stability and/or shelf-life of the components within the system 100.

The nucleophilic group-containing crosslinker component (e.g., trilysine acetate mixed with FD&C Blue, an amine-terminated polyethylene glycol mixed with methylene blue, etc.) 102a, 102b, 106a, 106b may be baked onto a basin 114a, 114b within recess 110a, 110b. As shown in FIG. 1, the nucleophilic group-containing crosslinker component 102a, 102b, 106a, 106b is in solid form. Basin 114a, 114b may be positioned within a well 112a, 112b of recess 110a, 110b. In certain embodiments, the nucleophilic group-containing crosslinker component 102a, 102b, 106a, 106b is not lyophilized. The nucleophilic group-containing crosslinker component 102a, 102b, 106a, 106b once baked may adhere to the basin with an adhesion force of about 1 μN to about 50 μN. Without a suitable adhesion strength, and/or if the nucleophilic component is too dry, the nulceophilic component may flake off of the container. In some embodiments, the nucleophilic group contains a small amount of water to enhance adhesion, for example, about 0.1 ppm by weight to about 100 ppm by weight, or any individual value or sub-range within these ranges.

In certain embodiments, ocular sealant system 100 may include an electrophilic group-containing multi-arm-polymer precursor component (e.g., containing units of a polyethylene glycol, such as, 8a15kSS) 103a, 103b. In certain embodiments, electrophilic group-containing multi-arm-polymer precursor component (e.g., containing units of a polyethylene glycol, such as, 8a15kSS) 103a, 103b is not lyophilized. A lyophilized electrophilic group-containing multi-arm-polymer precursor component containing units of a polyethylene glycol 300 according to the prior art is shown in FIG. 3A. The lyophilized electrophilic component 300 is in the form of a solid cake. To perform lyophilization, the polyethylene glycol component must be mixed with water and an HCl buffer to form a solution of having a pH of about 1.0 to about 5.0, about 3.8, or any individual pH or sub-range within this range. The lyophilized cake has cracks and recesses where water and solution can be trapped. The lyophilization process leaves residual water in the lyophilized cake, which can cause degradation and impact the stability of the components within the ocular sealant system.

In some embodiments, the electrophilic group-containing multi-arm-polymer precursor component may be heated to a melt and subsequently printed in a pattern along an elgonate portion 109a, 109b of recess 110a, 110b. FIG. 3B shows an electrophilic group-containing multi-arm-polymer precursor component (i.e., units of polyethylene glycol 8a15kSS) 303 according to the invention. After printing, the melted PEG is baked and dried to form a solid structure. Printing the electrophilic group-containing multi-arm-polymer precursor component does not require water thus reducing the water content of the electrophilic component and increasing the stability of the components within the ocular sealant system. Printing also enables the electrophilic component to be printed in a pattern, for example, small lines or dots, which increases the dissolution time of the electrophilic component when combined with the diluent and the nucleophilic component.

As shown in FIG. 1, the printed pattern may be a ladder comprised of lines of the electrophilic group-containing multi-arm-polymer precursor component. The ladder configuration may include spaced apart lines. In certain embodiments, the patterned configuration is formed of dots (not shown) instead of a ladder. The dots may have a diameter of about 1 μm to about 2,000 μm, about 5 μm to about 1,500 μm, about 10 μm to about 1,000 μm, or about 100 μm to about 500 μm, or any individual size or sub-range within these ranges. In some embodiments, each dot comprises about 1 μg to about 1,000 μg, about 5 μg to about 750 μg, or about 17 μg to about 500 μg, or any individual amount or sub-range within these ranges, of the electrophilic group-containing multi-arm-polymer precursor component. Each dot may be spaced apart from an adjacent dot by about 25 μm to about 2,000 μm, about 50 μm to about 1,000 μm, about 100 μm to about 500 μm or any individual distance or sub-range within these ranges.

Following printing, the electrophilic group-containing multi-arm-polymer precursor component may be baked and solidified onto the elongate portion 109a, 109b of recess 110a, 110b. The electrophilic group-containing multi-arm-polymer precursor component 103a, 103b, once baked, may adhere to the elongate portion 109a, 109b of the recess 110, 110b with an adhesion force of about 1 μN to about 50 μN. In certain embodiments, the patterned configuration provides a dissolution of the electrophilic group-containing multi-arm-polymer precursor component 103a, 103b into a solution in less than about 5 seconds.

The ocular sealant system 100 of FIG. 1, further includes a solvent bottle 114 configured to store a solvent 116 therein. The solvent bottle 114 may be a dropper bottle having a nozzle configured to dispense the solvent 116 dropwise. The terms “solvent bottle” or “dropper bottle” are broad terms that refer to a device that can deliver drops. Alternatives include bulbous droppers, pipettes and/or pipetting systems. Another embodiment is an ampoule with a cap that covers an opening sized to deliver its contents in small volumes, e.g., dropwise. In general, users can manually perform dropwise dispensing with good accuracy using a suitable dropper. Single-use droppers are generally convenient for purposes of sterility.

In certain embodiments, the solvent bottle 114 has a dispensing range of about 40 μL/drop to about 120 μL/drop, about 50 μL/drop to about 110 μL/drop, about 60 μL/drop to about 100 μL/drop, or about 64 μL/drop to about 96 μL/drop. The dropper bottle may be formed of an inert material such as a plastic or glass. Suitable plastics include, but are not limited to, plastic, polyethylene, low density polyethylene, high density polyethylene terephthalate, polytetrafluoroethylene, or combinations thereof. Suitable glass materials include, but are not limited to, borosilicate glass, phenolic glass, Flint glass, silica glass, or combinations thereof.

Ocular sealant system 100 further includes an applicator 117 having a delivery surface (or tip) 119 at an end thereof. The applicator 117 may be used to wet the delivery surface 119 in the ocular sealant formulation (i.e., once the individual components are combined) and to topically apply the formulation to the surface of the eye of a subject. The delivery surface 119 may be sized to hold a volume of no more than about 100 μl of a liquid volume, that is, dipping the surface into a body of pure distilled water and withdrawing the surface recovers no more than about 100 μl of the water. The term delivery surface 119 refers to a discrete surface in its entirety. In the context of a device, the delivery surface 119 can be understood as a sponge, pad or brush on the end of a mixing rod/applicator 117, or the portion that holds the volume when used as intended when transferring from a liquid to an eye. The term transfer in the context of use with a delivery surface 119 means that the volume that is held by the applicator 117 has to be substantially transferred, or transferable, to an eye, which is a delicate tissue. For the sake of clarity these terms are to be tested in the context of holding and transferring pure distilled water. Accordingly, a cotton ball will not substantially transfer the volume of water because it tends to absorb the water. On the other hand a small brush can hold the volume of water and transfer a greater portion of the volume.

A delivery surface 119 may be configured to provide a controllable amount of solution or gel (e.g., ocular sealant formulation) in a predetermined volume range so that the user can conveniently pick up as much solution as is reasonably needed; not too much and not too little. For instance, the delivery surface 119 can be sized and proportioned to provide a volume in a range or sub-range from about 5 microliters to about 500 microliters, about 5 microliters to about 100 microliters, from about 20 microliters to about 200 microliters, less than about 100 microliters, or less than about 50 microliters, or any individual volume or sub-range within these ranges. Such volume can be controlled by tapering, surface area and hydrophobicity of the tip in view of a density of the ocular sealant solution; for instance, a surface that is relatively hydrophobic can cause the solution to bead and form a drop as opposed to spreading more broadly on the surface to provide a larger drop size. The surface area of the delivery surface 119, and consequently the size of the surface, as well as its hydrophobicity control the size of the droplet volume that is naturally picked up. Some embodiments further control a size of droplet pickup by capillary forces created by including a feature on the applicator tip 117. Such feature may be, for example, a dimple, a crescent, a groove, a slit, a slot, an indentation or other feature.

For delicate tissues, e.g., a cornea, the applicator delivery surface 119 may be of a type that is atraumatic to the corneal or other delicate tissue surfaces, so as to not induce trauma, such as a corneal abrasion during normal application manipulation. A closed cell foam is suitable for the material of the applicator tip 119. Also suitable is a hydrophobic closed cell foam, such as a polyethylene foam. Another embodiment is a (soft) brush that is sized to hold a suitable volume without retaining the greater part of it. The applicator delivery surface 119 may be designed to not absorb a significant portion of liquid by itself, since this can create variability in the amount of material delivered to the application site. The amount needed to be delivered may be less than 10 microliters, which is smaller than one drop; for instance, opthalmic sealant applications typically require small volumes of materials. If the applicator delivery surface is made from a sponge or other material that absorb a significant amount of the liquid, then the application will have variability. Application of too much of a material, e.g., as in a hydrogel to the surface of the cornea, can create patient discomfort. Accordingly, delivery surfaces may be chosen that absorb less than about 30%, less than about 20%, less than about 10%, or essentially 0% of a solution's volume, including the case wherein the volume is less than about 100, less than about 50, less than about 20, or less than about 10 microliters. As is apparent, embodiments of the applicator 117 may have one or more of these features. The recessed dimples not only provide an area for the deposited components to be placed, but are also configured to produce turbulences in the solution while mixing.

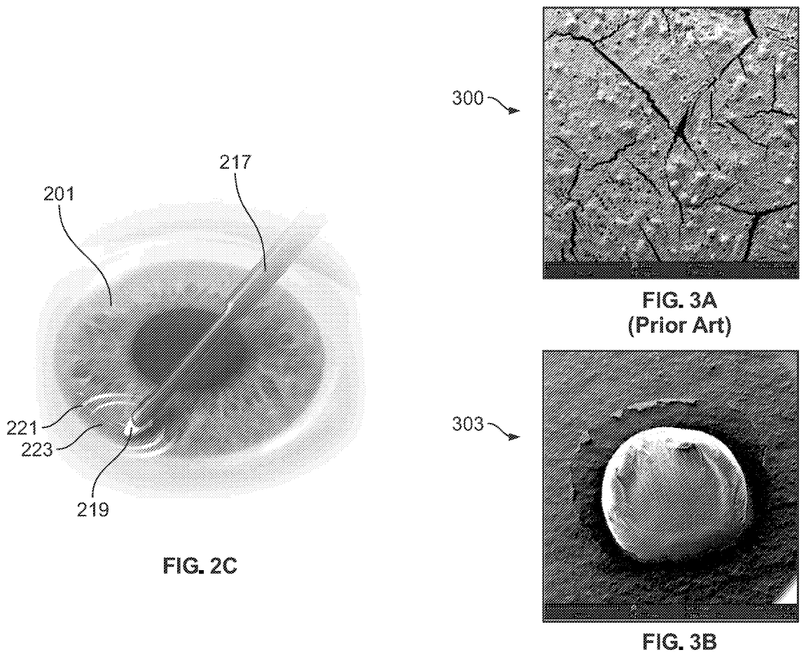

An embodiment of a container 208 for an ocular sealant system according to the present invention is shown in FIGS. 2A and 2B. One or more of the dimensions of the recesses 210a, 210b, placement of the components, quantity of components, molecular weight, functional groups and attribtues of the electrophilic group and patterning of the electrophilic group-containing multi-arm-polymer precursor component in the recesses can affect the dissolution and mixing time of the components when contacted with the solvent.

As shown in FIGS. 2A and 2B, container 208 includes two recesses 210a, 210b (e.g., identical in size and shape) configured to store both the nucleophilic group-containing crosslinker component and the electrophilic group-containing multi-arm-polymer precursor component and to hold these components together with the solvent when combining (e.g., dissolving and mixing) the components. In certain embodiments, the container 208 is formed of a high-density polyethylene, a polycarbonate, a polystyrene, a nylon, an aluminum foil or combinations thereof. In certain embodiments, the container 208 comprises less than about 0.50 wt. % water, less than about 0.25 wt. % water, less than about 0.20 wt. % water, or less than about 0.15 wt. % water based on the total weight of the container 208. In some embodiments, the container 208 includes a water absorbance value of less than about 0.50% water, less than about 0.25% water, less than about 0.20% water, or less than about 0.15% water. Reducing and/or eliminating water from the system may increase the stability and/or shelf-life of the components within the system.

An inner length 205 of each recess 210a, 210b may be about 20 mm to about 50 mm and an inner width 215 of the elongate portion may be about 3 mm to about 20 mm. In some embodiments, a ratio of the inner length 205 to the inner width 215 is about 2:1 to about 10:1, about 4:1 to about 7:1, or any individual ratio or sub-range within these ranges. In certain embodiments, the elongate portion along which the electrophilic group-containing multi-arm-polymer precursor component 203a, 203b is printed, has a width 216 of about 1 mm to about 5 mm. As shown in FIG. 2A, the electrophilic group-containing multi-arm-polymer precursor component 203a, 203b may be printed in a ladder pattern such that the length of each line is about the same as the width 216 of the elongate portion. The width 220 of each line in the ladder pattern may be about 0.1 mm to about 5.0 mm, about 0.5 mm to about 3.0 mm, about 0.85 mm to about 1.5 mm, or any individual width or sub-range within these ranges. The length 222 of the space between each line in the ladder pattern may be about 0.05 mm to about 4.0 mm, about 0.1 mm to about 3.0 mm, or about 0.61 mm to about 2.0 mm, or any individual length or sub-range within these ranges. In certain embodiments, there should be enough distance between individual lines of the ladder pattern to prevent joining of lines. The pattern, furthermore, should have sufficient space away from basin 214a, 214b to prevent undesired reaction between the nucleophilic group-containing crosslinker component 102a, 102b, 106a, 106b and the electrophilic group-containing multi-arm-polymer precursor component 103a, 103b during dry storage.

In certain embodiments, container 208 may have a total length 202 of about 30 mm to about 60 mm, or any individual length or sub-range within this range, and a total width 224 of about 30 mm to about 60 mm, or any individual length or sub-range within this range. Basin 114a, 114b may be positioned a length 206 of about 2 mm to about 10 mm from an end of the ladder pattern of the electrophilic group-containing multi-arm-polymer precursor component 203a, 203b. The ends of recesses 210a, 210b may be, independently, spaced from the edge of container 208 a length 213, 228 of about 0.5 mm to about 10 mm, or about 1.0 mm to about 5.0 mm, or any individual length or sub-range within these ranges.

A side view of container 208 is shown in FIG. 2B. Basin 214a, 214b is seated within a well 212a, 212b. The outer diameter 230 of well 212a, 212b about basin 214a, 214b may be about 3 mm to about 10 mm, about 4.0 mm to about 6.46 or any individual value or sub-range within this range. The diameter 232 of basin 214a, 214b may be about 1.0 mm to about 4.0 mm, or any individual value or sub-range within this range. The down angle 232 between well 212a, 212b and basin 214a, 214b may be about 1.0° to about 10°, about 4.38° to about 6.0°, or any individual angle or sub-range within these ranges. The angle 230 along well 212a, 212b may be about 5° to about 15°, about 10° to about 12°, or any individual angle or sub-range within these ranges. The radius 238 between the upper edge of basin 214a, 214b and bottom is about 1 mm to about 5 mm, or about 2.54 mm to about 3 mm, or any individual radius or sub-range within these ranges. The radius 240, 244 of the transition between basin 214a, 214b and well 212a, 212b may be about 1 mm to about 5 mm, or about 1.59 mm to about 3 mm, or any individual radius or sub-range within these ranges. The radius 242 between well 212a, 212b and recess 210a, 210b may be about 1 mm to about 5 mm, or about 1.27 mm to about 3 mm, or any individual radius or sub-range within these ranges.

As shown in FIG. 2C, once the components of the ocular sealant formulation are combined within a recess 210a of container 208, applicator 217 is used to submerge applicator tip 219 in the resultant formulation and to apply the ocular sealant formulation 223 to the eye 201 of a subject. As shown, the sealant formulation 223 is topically applied over a scratch or incision 221 on the surface of the eye 201.

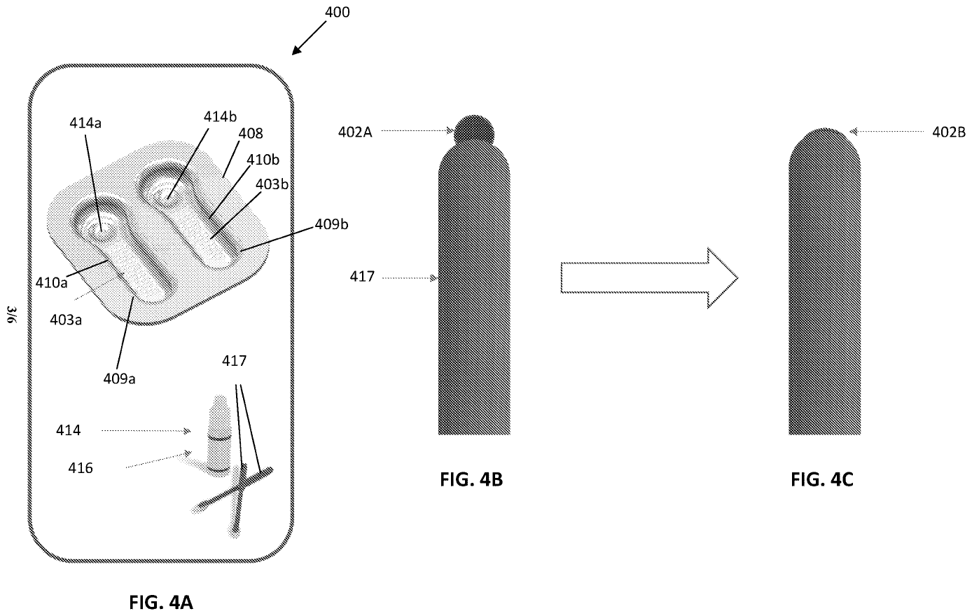

Another embodiment of an ocular sealant system 400 is shown in FIGS. 4A-4C. In this embodiment, container 408 has the same configuration and dimensions as container 108, 208 and is formed of the same materials. Container 408, however, does not have a nucleophilic group-containing crosslinker component baked onto basin 414a, 414b. Instead, the nucleophilic group-containing crosslinker component 402a, 402b (e.g., trilysine acetate, amine-terminated polyethylene glycol, etc.) is deposited onto an end of applicator 417. In certain embodiments, the nucleophilic component 402a is applied as a drop onto a tip end of the applicator 417 as shown in FIG. 4B. The drop 402a may be allowed to spread to form a thin film of the nucleophilic component 402b on the applicator 417. In some embodiments, drop 402a is heated or baked to cause the drop 402a to spread and form thin film 402b. In certain embodiments, the thin film of the nucleophilic component 402b is baked and dried onto the tip end of applicator 402b. The applicator 417 having the nucleophilic component thereon may then be sealed in a container (e.g., an inert plastic bag, a vial, etc.) (not shown). Because of the reduced volume, the concentration of the nucleophilic group-containing crosslinker component in thin film 402b may be higher than in a nucleophilic component deposited onto basin 414a, 414b. Depositing the nucleophilic component 402b on applicator 417 removes water from the container 408 manufacturing process as the nucleophilic component typically requires pre-dissolution of the component in water prior to deposition onto container 408. Additionally, separately packaging the applicator having the nucleophilic component thereon isolates the electrophilic component 403a, 403b from possible water contamination thus increasing the shelf-life of the components within system 400.

Ocular sealant system 400 further includes a solvent bottle 414, as described hereinabove, which holds and stores a solvent 416. Container 408, like containers 108, 208, has an electrophilic group-containing multi-arm-polymer precursor component 403a, 403b printed on an elongate portion 409a, 409b of recess 410a, 410b. The electrophilic component 403a, 403b may be printed in a pattern, e.g., small lines or dots, as illustrated by the ladder pattern in FIG. 4A. Solvent 416 may be added dropwise (or pipetted) into recess 410a, 410b whereupon contact, the electrophilic component 403a, 403b will begin to dissolve in the solvent 416. The tip end of applicator 417 having the thin film of nucleophilic component 402B deposited thereon may be submerged in the solvent 416 and electrophilic component 403a, 403b mixture. Applicator 417 may be used to mix the formulation within recess 410a, 410b and while mixing, the nucleophilic component 402b dissolves into the solution.

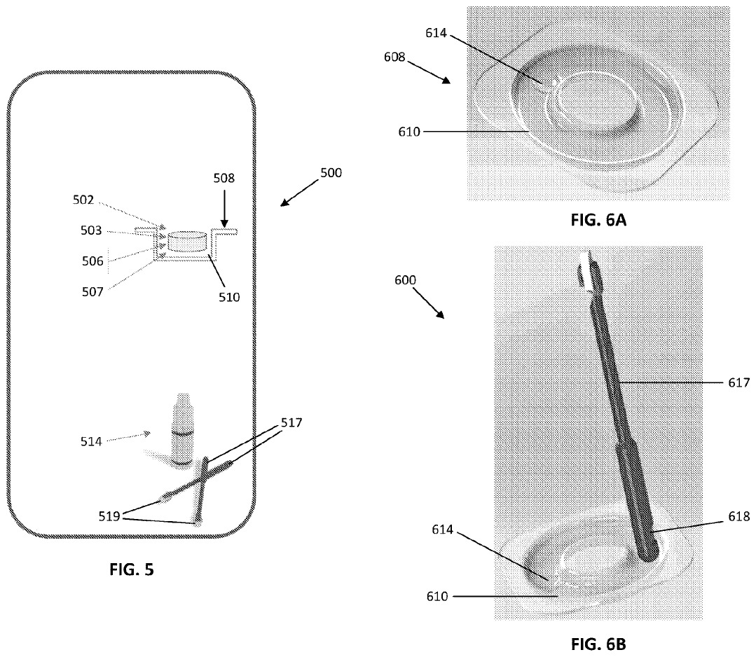

Another embodiment of an ocular sealant system 500 is shown in FIG. 5. In this embodiment, the ocular sealant formulation 507 is in the form of a solid dosage form (e.g., a tablet). Ocular sealant formulation 507 may be comprised of a nucleophilic group-containing crosslinker component 502, an electrophilic group-containing multi-arm-polymer precursor component 503 and one or more optional excipients 506 (e.g., a colorant, a solubility enhancer, etc.). In certain embodiments, each of components 502, 503, 506 may be independently milled, ground, crushed, granulated, etc., combined in desired amounts/ratios and compressed to form tablet 507. Tablet 507 may include a disintegrant, effervescent and/or solubility enhancer as described herein. Tablet 507 may be stored in recess 510 of container 508. A solvent stored in solvent bottle 514 may be added to recess 510 to dissolve tablet 507. Delivery surface 519 of applicator 517 may be submerged in the resulting ocular sealant formulation and topically applied to an eye of a subject.

In an alternative embodiment, rather than forming a tablet from the particles described above, the particles may be deposited on a tip end of applicator 517. For example, the particles may be compressed and adhered to applicator 517. In some embodiments, the particles may be suspended in a fluid that is applied dropwise to the tip end of applicator 517. In certain embodiments, once the particulate formulation is deposited on applicator 517, it may be heated and baked. To form the ocular sealant formulation, solvent may be added to the recess of the tray and the end of applicator 517 having the particular formulation may be submerged in the solvent, dissolved and then mixed.

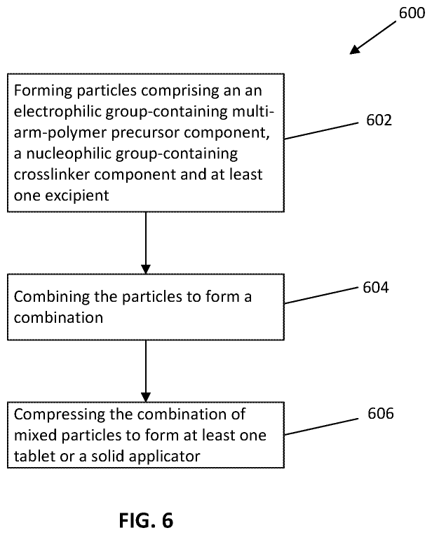

An embodiment of a container 608 for an ocular sealant formulation system 600 is shown in FIGS. 6A and 6B. FIG. 6B shows container 608 and an embodiment of an applicator 617 for use therewith. As shown in FIGS. 6A and 6B, container 608 can include a recess 610 on which the electrophilic component may be printed. Recess 610 may further include a basin 614 in which the nucleophilic component may be deposited. The circular shape of recess 610 may facilitate mixing of the electrophilic component and the nucleophilic component upon dissolution in the diluent. Applicator 617 may include notch 618 which may engage with an edge of recess 610 to assist in mixing the components with the diluent.

In some embodiments, the ocular sealant formulation may be formulated as a film (e.g., a thin film or laminate). The film can be formulated to contain the nucleophilic group, the electrophilic group and one or more excipient. After its preparation, the film can be applied directly to the surface of the eye, for example, over a wound. In some embodiments, the film can be cut to size and/or shape with a cutting implement (e.g., scissors, razor, blade, etc.), which may be sterile. Upon contact with the ocular surface, the film may form a hydrogel and the hydrogel will dissolve and/or resorb over time.

### The Polymer Network:

In certain embodiments, a hydrogel may be formed from precursors having functional groups that form crosslinks to create a polymer network. These crosslinks between polymer strands or arms may be chemical (i.e., may be covalent bonds) and/or physical (such as ionic bonds, hydrophobic association, hydrogen bridges etc.) in nature.

The polymer network may be prepared from precursors, either from one type of precursor or from two or more types of precursors that are allowed to react. Precursors are chosen in consideration of the properties that are desired for the resultant hydrogel. There are various suitable precursors for use in making the hydrogels. Generally, any pharmaceutically acceptable and crosslinkable polymers forming a hydrogel may be used for the purposes of the present invention. The hydrogel and thus the components incorporated into it, including the polymers used for making the polymer network, should be physiologically safe such that they do not elicit e.g. an immune response or substantial immune response or other adverse effects. Hydrogels may be formed from natural, synthetic, or biosynthetic polymers.

Natural polymers may include glycosaminoglycans, polysaccharides (e.g. dextran), polyaminoacids and proteins or mixtures or combinations thereof, while this list is not intended to be limiting.

Synthetic polymers may generally be any polymers that are synthetically produced from a variety of feedstocks by different types of polymerization, including free radical polymerization, anionic or cationic polymerization, chain-growth or addition polymerization, condensation polymerization, ring-opening polymerization, etc. The polymerization may be initiated by certain initiators, by light and/or heat, and may be mediated by catalysts. Synthetic polymers may in certain embodiments be used to lower the potential of allergies in dosage forms that do not contain any ingredients from human or animal origin.

Generally, for the purposes of the present invention one or more synthetic polymers of the group comprising one or more units of polyethylene glycol (PEG), polyethylene oxide, polypropylene oxide, polyvinyl alcohol, poly(vinylpyrrolidinone), polylactic acid, polylactic-co-glycolic acid, random or block copolymers or combinations/mixtures of any of these can be used, while this list is not intended to be limiting.

To form covalently crosslinked polymer networks, the precursors may be covalently crosslinked with each other. In certain embodiments, precursors with at least two reactive centers (for example, in free radical polymerization) can serve as crosslinkers since each reactive group can participate in the formation of a different growing polymer chain.

The precursors may have biologically inert and hydrophilic portions, e.g., a core. In the case of a branched polymer, a core refers to a contiguous portion of a molecule joined to arms that extend from the core, where the arms carry a functional group, which is often at the terminus of the arm or branch. Multi-armed PEG precursors are examples of such precursors and are used in particular embodiments of the present invention as further disclosed herein.

A hydrogel for use in the present invention can be made e.g. from one multi-armed precursor with a first (set of) functional group(s) and another (e.g. multi-armed) precursor having a second (set of) functional group(s). By way of example, a multi-armed precursor may have hydrophilic arms, e.g., polyethylene glycol units, terminated with primary amines (nucleophile), or may have activated ester end groups (electrophile). The polymer network according to the present invention may contain identical or different polymer units crosslinked with each other. The precursors may be high-molecular weight components (such as polymers having functional groups as further disclosed herein) or low-molecular weight components (such as low-molecular amines, thiols, esters, carboxyls, carbonyls, etc. as also further disclosed herein).

Certain functional groups can be made more reactive by using an activating group. Such activating groups include (but are not limited to) carbonyldiimidazole, sulfonyl chloride, aryl halides, sulfosuccinimidyl esters, N-hydroxysuccinimidyl (abbreviated as “NHS”) ester, succinimidyl ester, benzotriazolyl ester, thioester, epoxide, aldehyde, maleimides, imidoesters, acrylates and the like. The NHS esters are useful groups for crosslinking with nucleophilic polymers, e.g., primary amine-terminated or thiol-terminated polyethylene glycols. An NHS-amine crosslinking reaction may be carried out in aqueous solution and in the presence of buffers, e.g., phosphate buffer (pH 5.0-7.5), triethanolamine buffer (pH 7.5-9.0), borate buffer (pH 9.0-12), or sodium bicarbonate buffer (pH 9.0-10.0).

In certain embodiments, each precursor may comprise only nucleophilic or only electrophilic functional groups, so long as both nucleophilic and electrophilic precursors are used in the crosslinking reaction. Thus, for example, if a crosslinker has only nucleophilic functional groups such as amines, the precursor polymer may have electrophilic functional groups such as N-hydroxysuccinimides. On the other hand, if a crosslinker has electrophilic functional groups such as sulfosuccinimides, then the functional polymer may have nucleophilic functional groups such as amines or thiols. Thus, functional polymers such as proteins, poly (allyl amine), or amine-terminated di- or multifunctional poly(ethylene glycol) can be also used to prepare the polymer network of the present invention.

In one embodiment of the present invention a precursor for the polymer network forming the hydrogel has about 2 to about 16 nucleophilic functional groups each (termed functionality), and in another embodiment a precursor has about 2 to about 16 electrophilic functional groups each (termed functionality). Reactive precursors having a number of reactive (nucleophilic or electrophilic) groups as a multiple of 4, thus for example 4, 8 and 16 reactive groups, are particularly suitable for the present invention. However, any number of functional groups, such as including any of 2, 3, 4, 5, 6, 7, 8, 9, 10, 11, 12, 13, 14, 15, or 16 groups, is possible for precursors to be used in accordance with the present invention, while ensuring that the functionality is sufficient to form an adequately crosslinked network.

### PEG Hydrogels:

In certain other embodiments, the crosslinking agent used is a low-molecular weight component containing nucleophilic end groups, such as amine or thiol end groups. In certain embodiments, the nucleophilic group-containing crosslinking agent is a small molecule amine with a molecular weight below about 100,000 Da, below about 50,000 Da, below about 25,000 Da, below about 10,000 Da, or below about 1,000 Da, or any individual value or sub-range within these ranges, comprising two or more primary aliphatic amine groups. A particular crosslinking agent for use in the present invention is, e.g., dilysine, trilysine, tetralysine, ethylenediamine, 1,3-diaminopropane, 1,3-diaminopropane, diethylenetriamine, trimethylhexamethylenediamine, amine-terminated polyethylene glycol, their pharmaceutically acceptable salts, hydrates, and derivatives such as conjugates (as long as sufficient nucleophilic groups for crosslinking remain present), and any mixtures thereof. In certain preferred embodiments, trilysine is used as crosslinking agent. It is understood that trilysine as used herein refers to trilysine in any form including a trilysine salt, such as trilysine acetate or a trilysine derivative such as a labeled trilysine.

In certain embodiments, electrophilic end groups for use with PEG precursors for preparing the hydrogels of the present invention are N-hydroxysuccinimidyl (NHS) esters, including but not limited to NHS dicarboxylic acid esters such as the succinimidylmalonate group, succinimidylmaleate group, succinimidylfumarate group, “SAZ” referring to a succinimidylazelate end group, “SAP” referring to a succinimidyladipate end group, “SG” referring to a succinimidylglutarate end group, and “SS” referring to a succinimidylsuccinate end group.

In certain embodiments of the present invention, the polymer network forming the hydrogel contains an electrophilic group-containing multi-arm-polymer precursor component comprised of, for example, polyethylene glycol units. PEGs are known in the art to form hydrogels when crosslinked, and these PEG hydrogels are suitable for pharmaceutical applications, e.g., as matrix for drugs intended to be administered to any part of the human or animal body.

The polymer network of the hydrogel of the present invention may comprise one or more multi-arm PEG units having from 2 to 10 arms, or from 4 to 8 arms, or 4, 5, 6, 7 or 8 arms. In certain embodiments, the PEG units used in the hydrogel of the present invention have 4 arms. In certain embodiments, the PEG units used in the hydrogel of the present invention have 8 arms. In certain embodiments, PEG units having 4 arms and PEG units having 8 arms are used in the hydrogel of the present invention. In certain particular embodiments, one or more 4-armed PEGs is/are utilized.

The number of arms in the PEG contributes to controlling the flexibility and/or softness of the resulting hydrogel. For example, hydrogels formed by crosslinking 8-arm PEGs are generally harder and less flexible than those formed from 4-arm PEGs of the same molecular weight. In particular, if a more flexible hydrogel is desired, the hydrogel may be formed using a 4-arm PEG, optionally in combination with another multi-arm PEG, such as an 8-arm PEG as disclosed above, or another (different) 4-arm PEG. If a stiffer hydrogel is desired, the hydrogral may be formed using an 8-arm PEG alone or in combination with another PEG component.

In certain embodiments of the present invention, polyethylene glycol units used as precursors have an average molecular weight in the range from about 2,000 to about 100,000 Daltons, or in a range from about 10,000 to about 60,000 Daltons, or in a range from about 15,000 to about 50,000 Daltons, or any individual value or sub-range within these ranges. In certain particular embodiments the polyethylene glycol units have an average molecular weight in a range from about 10,000 to about 40,000 Daltons, or any individual value or sub-range within these ranges. In specific embodiments, the polyethylene glycol units used for making the hydrogels according to the present invention have an average molecular weight of about 15,000 Daltons. Polyethylene glycol precursors of different molecular weight may be combined with each other. When referring herein to a PEG material having a particular average molecular weight, such as about 15,000 Daltons, a variance of ±10% is intended to be included, i.e., referring to a material having an average molecular weight of about 15,000 Daltons also refers to such a material having an average molecular weight of about 13,500 to about 16,500 Daltons. As used herein, the abbreviation “k” in the context of the molecular weight refers to 1,000 Daltons, i.e., “15k” means 15,000 Daltons.

In an 8-arm (“8a”) PEG, in certain embodiments each of the arms may have an average arm length (or molecular weight) of the total molecular weight of the PEG divided by 8. A 8a15kSS precursor, which is a particularly suitably precursor for use in the present invention thus has 8 arms with an average molecular weight of about 1,875 Daltons each and a total molecular weight of 15,000 Daltons. An 8a15kSS PEG precursor, which could also be used in combination with, e.g., a 4a20kPEG precursor in the present invention, thus has 4 arms (“4a”) each having an average molecular weight of 5,000 Daltons and a total molecular weight of 20,000 Daltons. Longer arms may provide increased flexibility as compared to shorter arms. PEGs with longer arms may swell more as compared to PEGs with shorter arms. A PEG with a lower number of arms also may swell more and may be more flexible than a PEG with a higher number of arms. In certain particular embodiments, only an 8-arm PEG precursor is utilized in the present invention. In certain other embodiments, a combination of an 8-arm PEG precursor and a 4-arm precursor is utilized in the present invention. In addition, longer PEG arms have higher melting temperatures when dry, which may provide more dimensional stability during storage.

In certain embodiments, electrophilic end groups for use with PEG precursors for preparing the hydrogels of the present invention are N-hydroxysuccinimidyl (NHS) esters, including but not limited to NHS dicarboxylic acid esters such as the succinimidylmalonate group, succinimidylmaleate group, succinimidylfumarate group, “SAZ” referring to a succinimidylazelate end group, “SAP” referring to a succinimidyladipate end group, “SG” referring to a succinimidylglutarate end group, and “SS” referring to a succinimidylsuccinate end group.

In certain embodiments, nucleophilic end groups for use with electrophilic group-containing PEG precursors for preparing the hydrogels of the present invention are amine (denoted as “NH₂”) end groups. Thiol (—SH) end groups or other nucleophilic end groups are also possible.

In certain embodiments of the present invention, 8-arm PEGs with an average molecular weight of about 15,000 Daltons and electrophilic end groups as disclosed above (such as the SAZ, SAP, SG and SS end groups, particularly the SS end group) are crosslinked for forming the polymer network and thus the hydrogel according to the present invention. Suitable PEG precursors are available from a number of suppliers, such as Jenkem Technology and others.

Reactions of e.g. nucleophilic group-containing crosslinkers and electrophilic group-containing PEG units, such as reaction of amine group-containing crosslinkers with activated ester-group containing PEG units, result in a plurality of PEG units being crosslinked by a hydrolyzable linker having the formula:

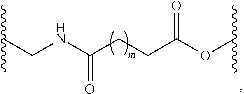

wherein m is an integer from 0 to 10, and specifically is 1, 2, 3, 4, 5, 6, 7, 8, 9, or 10. For a SAZ-end group, m would be 6, for a SAP-end group, m would be 3, for a SG-end group, m would be 2 and for an SS-end group, m would be 1.

In certain embodiments, the polymer precursors used for forming the hydrogel according to the present invention may be selected from 4a20kPEG-SAZ, 4a20kPEG-SAP, 4a20kPEG-SG, 4a20kPEG-SS, 8a20kPEG-SAZ, 8a20kPEG-SAP, 8a20kPEG-SG, 8a20kPEG-SS, or mixtures thereof, with one or more PEG- or lysine based-amine groups selected from 4a20kPEG-NH₂, 8a20kPEG-NH₂, and trilysine, or a trilysine salt or derivative, such as trilysine acetate.

In certain embodiments, the SS end group is utilized in the present invention. This end group may provide for a shorter time until the hydrogel is biodegraded in an aqueous environment such as in the tear fluid, when compared to the use of other end groups, such as the SAZ end group, which provides for a higher number of carbon atoms in the linker and may thus be more hydrophobic and therefore less prone to ester hydrolysis than the SS end group.

In particular embodiments, an 8-arm 15,000 Dalton PEG precursor having a SS end group (as defined above), is crosslinked with a crosslinking agent having one or more reactive amine end groups. This PEG precursor is abbreviated herein as 8a15kPEG-SS. A schematic chemical structure of 8a15kPEG-SS is reproduced below:

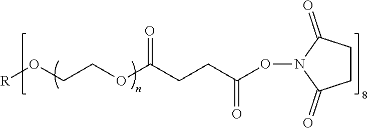

In this formula, n is determined by the molecular weight of the respective PEG-arm.

In certain particular embodiments, the crosslinking agent (herein also referred to as “crosslinker”) used is a low-molecular weight component containing nucleophilic end groups, such as amine or thiol end groups. In certain embodiments, the nucleophilic group-containing crosslinking agent is a small molecule amine with a molecular weight below 30,000 Da. In certain embodiments, the nucleophilic-group containing crosslinking agent comprises two, three or more primary aliphatic amine groups. Suitable crosslinking agents for use in the present invention are (without being limited to) spermine, spermidine, lysine, dilysine, trilysine, tetralysine, polylysine, ethylenediamine, polyethylenimine, 1,3-diaminopropane, 1,3-diaminopropane, diethylenetriamine, trimethylhexamethylenediamine, 1,1,1-tris(aminoethyl)ethane, amine-terminated polyethylene glycol. their pharmaceutically acceptable salts, hydrates or other solvates and their derivatives such as conjugates (as long as sufficient nucleophilic groups for crosslinking remain present), and any mixtures thereof. A particular crosslinking agent for use in the present invention is trilysine or a trilysine salt or derivative, such as trilysine acetate. Other low-molecular weight multi-arm amines may be used as well. The chemical structure of trilysine is reproduced below:

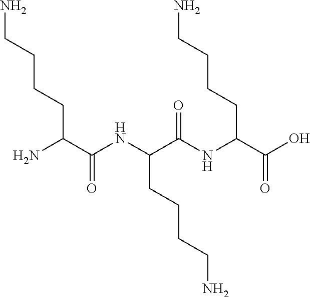 [SMILES: NCCCCC(N)C(=O)NC(CCCCN)C(=O)NC(CCCCN)C(=O)O]

In very particular embodiments of the present invention, an 8a15kSS precursor is reacted with trilysine acetate, to form the polymer network.

Another particular crosslinking agent for use in the present invention is 8a20kNH2. Other low-molecular weight multi-arm amine terminated polyethylene glycols may be used as well. The chemical structure of 8a20kNH2 is reproduced below:

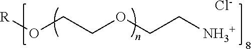

In this formula, n is determined by the molecular weight of the respective PEG-arm.

In very particular embodiments of the present invention, an 8a15kSS precursor is reacted with 8a20kNH2, to form the polymer network.

In certain embodiments, the nucleophilic group-containing crosslinking agent is bound to or conjugated with a visualization agent. Fluorophores such as fluorescein, rhodamine, coumarin, and cyanine can be used as visualization agents as disclosed herein. In specific embodiments of the present invention, fluorescein is used as the visualization agent. The visualization agent may be conjugated with the crosslinking agent e.g. through some of the nucleophilic groups of the crosslinking agent. Since a sufficient amount of the nucleophilic groups are necessary for crosslinking, “conjugated” or “conjugation” in general includes partial conjugation, meaning that only a part of the nucleophilic groups are used for conjugation with the visualization agent, such as about 1% to about 20%, or about 5% to about 10%, or about 8% of the nucleophilic groups of the crosslinking agent may be conjugated with a visualization agent. In specific embodiments, the crosslinking agent is trilysine acetate and is conjugated with fluorescein.

In other embodiments, the visualization agent may also be conjugated with the polymer precursor, e.g. through certain reactive (such as electrophilic) groups of the polymer precursors. In certain embodiments, the crosslinking agent itself or the polymer precursor itself may contain an e.g. fluorophoric or other visualization-enabling group.

In the present invention, conjugation of the visualization agent to either the polymer precursor(s) or to the crosslinking agent as disclosed below is intended to keep the visualization agent in the hydrogel while the active agent is released into the tear fluid.

In some embodiments, the nucleophilic group-containing crosslinker component (e.g., 8a20kNH2, trilysine acetate, etc.) is combined with a colorant. Suitable colorants include, but are not limited to, FD&C Blue No. 1 (Brilliant Blue FCF), FD&C Blue No. 2 (Indigotine), methylene blue, FD&C Green No. 3 (Fast Green FCF), FD&C Red No. 3 (Erythrosine), FD&C Yellow No. 5 (Tartrazine), and/or FD&C Yellow No. 6 (Sunset Yellow). When the nucleophilic group-containing crosslinker component is combined with the electrophilic group-containing multi-arm-polymer precursor component (e.g., units of polyethylene glycol) and dissolved together in the solvent, the colorant shows whether the nucleophilic group-containing crosslinker component has been completely and/or homogenously dissolved within the resulting solution.

In certain embodiments, the molar ratio of the nucleophilic and the electrophilic end groups reacting with each other is about 2:1, i.e., one amine group is provided per one electrophilic, such as SS, group. In the case of 8a15kSS and trilysine (acetate) this results in a molar ratio of the two components of about 2:1 as the trilysine has four primary amine groups that may react with the electrophilic SS ester group. However, an excess of either the electrophilic (e.g, the NHS end groups, such as the SS) end group precursor or of the nucleophilic (e.g, the amine) end group precursor may be used. In particular, an excess of the nucleophilic, such as the amine end group containing precursor or crosslinking agent may be used. In certain embodiments, the molar ratio of the electrophilic group containing precursor to the nucleophilic group-containing crosslinking agent, such as the molar ratio of 8a15kPEG-SS to trilysine acetate, is from about 1:2 to about 0.5:1, or from about 1:2 to about 2:1.

Finally, in alternative embodiments the amine linking agent can also be another PEG precursor with the same or a different number of arms and the same or a different arm length (average molecular weight) as the 8a15kPEG-SS, but having terminal amine groups, i.e., 8a20kPEG-NH₂.

### Active Agents:

The ocular sealant formulations and/or dosage forms of the present invention may contain, in addition to the polymer units forming the polymer network as disclosed above, active agents. Indeed, the hydrogel may be used to deliver classes of drugs including steroids, non-steroidal anti-inflammatoires (NSAIDS), intraocular pressure lowering drugs, antibiotics, pain relievers, inhibitors or vascular endothelial growth factor (VEGF), chemotherapeutics, antiviral drugs and combinations of any two or more of the foregoing. The drugs themselves may be small molecules, proteins, RNA fragments, proteins, glycosaminoglycans, carbohydrates, nucleic acid, inorganic and organic biologically active compounds where specific biologically active agents include but are not limited to: enzymes, antibiotics, antineoplastic agents, local anesthetics, hormones, angiogenic agents, anti-angiogenic agents, growth factors, antibodies, neurotransmitters, psychoactive drugs, anticancer drugs, chemotherapeutic drugs, drugs affecting reproductive organs, genes, and oligonucleotides, or other configurations. The drugs that have low water solubility may be incorporated, e.g., as particulates or as a suspension. Higher water solubility drugs may be loaded within microparticles or liposomes. Microparticles can be formed from, e.g., PLGA, fatty acids or any other suitable polymers.

Suitable NSAIDS include, but are not limited to, ibuprofen, meclofenamate sodium, mefanamic acid, salsalate, sulindac, tolmetin sodium, ketoprofen, diflunisal, piroxicam, naproxen, etodolac, flurbiprofen, fenoprofen calcium, Indomethacin, celoxib, ketrolac, and nepafenac.

In certain embodiments, the ocular sealant formulations and/or dosage forms include an active agent comprising a cyclodextrin. Suitable cyclodextrins include, but are not limited to, a hexasaccharide derived from glucose, an alpha-cyclodextrin, a beta-cyclodextrin, a gamma-cyclodextrin, methyl-substituted cyclodextrin, ethyl-substituted cyclodextrin, hydroxyalkyl-substituted cyclodextrins including 2-hydroxypropyl-beta-cyclodextrin, alkyl ether cyclodextrins, branched cyclodextrins, cationic cyclodextrins, quaternary ammonium cyclodextrins, anionic cyclodextrins, amphoteric cyclodextrins, sulfoalkyl ether beta-cyclodextrin or modified forms thereof and/or combination thereof. Suitable alpha-cyclodextrins include, but are not limited to, hydroxypropyl alpha cyclodextrin, hydroxybutyl alpha cyclodextrin, sulfobutyl alpha cyclodextrin, sulfopropyl alpha cyclodextrin, carboxyethyl alpha cyclodextrin, succinyl alpha cyclodextrin and succinylhydroxypropyl alpha cyclodextrin, modified forms thereof and/or combinations thereof. Suitable beta-cyclodextrins include, but are not limited to, a cyclic oligosaccharide consisting of seven glucose subunits joined by α-(1,4) glycosidic bonds, a hydroxyalkyl-beta-cyclodextrin, a sulfoalkyl ether-beta-cyclodextrin, hydroxypropyl-beta-cyclodextrin, sulfobutyl ether-beta-cyclodextrin, modified forms thereof and/or combinations thereof. Suitable gamma-cyclodextrins include, but are not limited to, a cyclic alpha-(1,4)-linked oligosaccharide consisting of eight glucose molecules, 6-per-deoxy-6-per-halo-gamma-cyclodextrin, modified forms thereof and/or combinations thereof.

In certain embodiments, the ocular sealant formulations and/or dosage forms include an active agent comprising an antibiotic. Suitable antibiotics include, but are not limited to, a protein, azithromycin, bacitracin, neomycin, polymyxin B, mupirocin, nitrofurazone, fusidic acid or combinations thereof. In certain embodiments, the ocular sealant formulations and/or dosage forms may include an active agen suitable for treating glaucoma. Such active agents include, but are not limited to, acetazolamide, anandamide, a cannabinoid, cyclosporin, a dehydroepiandrosterone, dexamethasone, diclofenac, dipivefrine, fluorometholone, hydrocortisone, loteprednol etabonate or pilocarpine.

A variety of drugs or other therapeutic agents may be delivered using these systems. A list of agents or families of drugs and examples of indications for the agents are provided. The agents may also be used as part of a method of treating the indicated condition or making a composition for treating the indicated condition. For example, AZOPT® (a brinzolamide opthalmic Suspension) may be used for treatment of elevated intraocular pressure in patients with ocular hypertension or open-angle glaucoma. BETADINE® in a Povidone-iodine ophthalmic solution may be used for prepping of the periocular region and irrigation of the ocular surface. BETOPTIC® (betaxolol HCl) may be used to lower intraocular pressure, or for chronic open-angle glaucoma and/or ocular hypertension. CILOXAN® (Ciprofloxacin HCl opthalmic solution) may be used to treat infections caused by Susceptible strains of microorganisms. NATACYN® (Natamycin opthalmic suspension) may be used for treatment of fungal blepharitis, conjunctivitis, and keratitis. NEVANAC® (Nepanfenac opthalmic suspension) may be used for treatment of pain and inflammation associated with cataract surgery. TRAVATAN® (Travoprost ophthalmic solution) may be used for reduction of elevated intraocular pressure—open-angle glaucoma or ocular hypertension. FML FORTE® (Fluorometholone ophthalmic suspension) may be used for treatment of corticosteroid-responsive inflammation of the palperbral and bulbar conjunctiva, cornea and anterior segment of the globe. LUMIGAN® (Bimatoprost ophthalmic solution) may be used for reduction of elevated intraocular pressure—open-angle glaucoma or ocular hypertension. PRED FORTE® (Prednisolone acetate) may be used for treatment of steroid-responsive inflammation of the palpebral and bulbar conjunctiva, cornea and anterior segment of the globe. PROPINE® (Dipivefrinhydrochloride) may be used for control of intraocular pressure in chronic open-angle glaucoma. RESTASIS® (Cyclosporine ophthalmic emulsion) may be used to increases tear production in patients, e.g., those with ocular inflammation associated with keratoconjunctivitis sicca. ALREX® (Loteprednoletabonate ophthalmic suspension) may be used for temporary relief of seasonal allergic conjunctivitis. LOTEMAX® (Loteprednol etabonate ophthalmic suspension) may be used for treatment of steroid responsive inflammation of the palpebral and bulbar conjunctiva, cornea and anterior segment of the globe. MACUGEN® (Pegaptainib sodium injection) may be used for Treatment of neovascular (wet) age-related macular degeneration. OPTIVAR® (AZelastine hydrochloride) may be used for treatment of itching of the eye associated with allergic conjunctivitis. XALATAN® (Latanoprost ophthalmic solution) may be used to reduce elevated intraocular pressure in patients, e.g., with open-angle glaucoma or ocular hypertension. BETIMOL® (Timolol opthalmic solution) may be used for treatment of elevated intraocular pressure in patients with ocular hypertension or open-angle glaucoma. Latanoprost is the pro-drug of the free acid form, which is a prostanoid selective FP receptor agonist. Latanoprost reduces intraocular pressure in glaucoma patients with few side effects. Latanoprost has a relatively low solubility in aqueous solutions, but is readily soluble in organic solvents typically employed for fabrication of microspheres using solvent evaporation.

One embodiment comprises extended release of a medication for allergic conjunctivitis. For instance, ketotifen, an antihistamine and mast cell stabilizer, may be released to the eye as described herein in effective amounts to treat allergic conjunctivitis. Seasonal Allergic Conjunctivitis (SAC) and Perennial Allergic Conjunctivitis (PAC) are allergic conjunctival disorders. Symptoms include itching and pink to reddish eyes. These two eye conditions are mediated by mast cells. Non specific measures to ameliorate symptoms convention ally include: cold compresses, eyewashes with tear Substitutes, and avoidance of allergens. Treatment conventionally consists of antihistamine mast cell stabilizers, dual mechanism anti-allergen agents, or topical antihistamines. Corticosteroids might be effective but, because of side effects, are reserved for more severe forms of allergic conjunctivitis such as Vernal keratoconjunctivitis (VKC) and atopic keratoconjunctivitis (AKC).

Moxifloxacin is the active ingredient in VIGAMOX®, which is a fluoroquinolone approved for use to treat or prevent ophthalmic bacterial infections. Dosage is typically one-drop of a 0.5% solution that is administered 3 times a day for a period of one-week or more.

VKC and AKC are chronicallergic diseases where eosinophils, conjunctival fibroblasts, epithelial cells, mast cells, and/or TH2 lymphocytes aggravate the biochemistry and histology of the conjunctiva. VKC and AKC can be treated by medications used to combat allergic conjunctivitis.

Accordingly, embodiments include hydrogels that incorporate one or more of the agents. The agents may be incorporated using one or more processes herein, e.g., with or without microspheres. The hydrogels may be used to make medicaments for administration of an effective amount of the agent over a predetermined time to treat the conditions indicated.

Some therapeutic agents are visualization agents as discussed above.

### Additional Ingredients:

The ocular sealant formulations and/or dosage forms of the present invention may contain, in addition to the polymer units forming the polymer network as disclosed above, other additional ingredients. Such additional ingredients are for example salts originating from buffers used during the preparation of the hydrogel, such as phosphates, borates, bicarbonates, or other buffer agents such as triethanolamine. In certain embodiments of the present invention sodium phosphate buffers (specifically, mono- and dibasic sodium phosphate) are used. Such buffers, may be dissolved within the solvent (e.g., an aqueous solvent).

In some embodiments, one or more components of the ocular sealant formulations and/or dosage forms contains a preservative. The preservative may be present in the formulations or dosage forms at a concentration of about 0.005 wt % to about 0.1 wt %, about 0.02 wt % to about 0.04 wt % based on the total weight of the formulation and/or dosage form. Suitable preservatives for ocular formulations include, but are not limited to, a quaternary ammonium compound such as benzalkonium chloride (i.e., N-benzyl-N—(C₈-C₁₈ alkyl)-N,N-dimethylammonium chloride), benzoxonium chloride, polyquaternium-1, polyquaternium-42, cetrimide, or the like; antioxidants such as vitamin A, vitamin E, vitamin C, retinyl palmitate, and selenium; oxidizing agents such as sodium perborate and stabilized oxochloro complex; the amino acids cysteine and methionine; an amidine such as chlorhexidine; citric acid and sodium citrate; ionic buffers such as borate, sorbitol, propylene glycol and zinc; mercury-based such as thimerosal and phenylmercuric nitrate/acetate; alkyl parabens such as methyl paraben and propyl paraben; octadecyldimethylbenzyl ammonium chloride, hexamethonium chloride, benzethonium chloride, phenol, catechol, resorcinol, cyclohexanol, 3-pentanol, m-cresol, phenylmercuric nitrate, phenylmercuric acetate or phenylmercuric borate, sodium chlorite, alcohols, such as chlorobutanol, butyl or benzyl alcohol or phenyl ethanol, guanidine derivatives, such as chlorohexidine or polyhexamethylene biguanide, sodium perborate, diazolidinyl urea, sorbic acid and/or combinations thereof.

While preservatives in multi-dose formulations of topical ophthalmic medications help maintain sterility, they can be toxic to the ocular surface. For example, benzalkonium chloride (BAK)—used in approximately 70% of ophthalmic formulations—causes cytotoxic damage to conjunctival and corneal epithelial cells, resulting in signs and symptoms of ocular surface disease (OSD) including ocular surface staining, increased tear break-up time, and higher OSD symptom scores. These adverse effects are more problematic with chronic exposure, as in lifetime therapy for glaucoma, but can also manifest after exposure as brief as 7 days.

According to at least one embodiment, the ocular sealant formulations and/or dosage forms include a preservative other than a quarternary ammonium compound, such as benzalkonium chloride, or the formulation and/or dosage form is preservative-free. In embodiments, the formulation and/or dosage form utilized in the present invention is preservative-free or at least does not contain a substantial amount of preservative. For example, the formulation and/or dosage form may contain less than about 0.005 wt %, less than about 0.001 wt %, or 0 wt % of preservative. The formulation and/or dosage form as described herein may be free of any one or more of the preservatives described above. In some embodiments, the formulation and/or dosage form is free of a quarternary ammonium compound. For example, the formulation and/or dosage form is free of benzalkonium chloride.

Preservative-free formulations and/or dosage forms that form hydrogrels according to embodiments herein can provide a suitable sustained release drug delivery platform for treating infection. Hydrogels can be formulated as biocompatible hydrophilic cross-linked polymer networks that swell when exposed to water. The antibiotic can be incorporated into the polymer matrix without preservatives. The rate of drug delivery, and duration of action of a hydrogel-based therapeutic, may be determined by the degree of polymeric crosslinking and the relative sizes of the inter-crosslink mesh openings and the drug to be delivered. An antibiotic formulation/dosage form may be a preservative-free formulation of about 0.1 mg to about 10 mg of antibiotic in a hydrogel sustained-release delivery system to treat infection. As it dissolves on the surface of the eye, the ocular sealant formulation delivers a tapering dose of antibiotic to the surface of the eye for up to 30 days.

In a further specific embodiment, the formulations and/or dosage forms utilized in the present invention do not contain any ingredients of animals or human origin. In some embodiments, the formulations and/or dosage forms contain only synthetic ingredients.

In certain embodiments, the ocular sealant formulations and/or dosage forms utilized in the present invention contain a visualization agent. Visualization agents to be used according to the present invention are all agents that can be conjugated with the components of the hydrogel or can be entrapped within the hydrogel, and that are visible, or may be made visible when exposed e.g. to light of a certain wavelength, or that are contrast agents. Suitable visualization agents for use in the present invention are (but are not limited to) e.g. fluoresceins, rhodamines, coumarins, cyanines, europium chelate complexes, boron dipyromethenes, benzofrazans, dansyls, bimanes, acridines, triazapentalenes, pyrenes and derivatives thereof. Such visualization agents are commercially available e.g. from TCI. In certain embodiments the visualization agent is a fluorophore, such as fluorescein or comprises a fluorescein moiety. Visualization of the fluorescein-containing formulation/dosage form is possible by illumination with blue light. The fluorescein in the ocular sealant formulation and/or dosage form illuminates when excited with blue light enabling confirmation of homogenous dissolution of the nucleophilic group-containing crosslinker component. In specific embodiments, the visualization agent is conjugated with one of the components forming the hydrogel. For example, the visualization agent, such as fluorescein, is conjugated with the crosslinking agent, such as the trilysine or trilysine salt or derivate (e.g, the trilysine acetate), or with the PEG-component e.g. by means of reacting NHS-fluorescein with trilysine acetate. Conjugation of the visualization agent prevents the visualization agent from being eluted or released out of the formulation/dosage form. Since a sufficient amount of the nucleophilic groups (at least more than one molar equivalent) are necessary for crosslinking, partial conjugation of the visualization agent with e.g, the crosslinking agent as disclosed above may be performed.

In some embodiments, the nucleophilic group-containing crosslinker component (e.g., 8a20kNH2, trilysine acetate, etc.) is combined with a colorant. Suitable colorants include, but are not limited to, FD&C Blue No. 1 (Brilliant Blue FCF), FD&C Blue No. 2 (Indigotine), methylene blue, FD&C Green No. 3 (Fast Green FCF), FD&C Red No. 3 (Erythrosine), FD&C Yellow No. 5 (Tartrazine), and/or FD&C Yellow No. 6 (Sunset Yellow). When the nucleophilic group-containing crosslinker component is combined with the electrophilic group-containing multi-arm-polymer precursor component (e.g., units of polyethylene glycol) and dissolved together in the solvent, the colorant shows whether the nucleophilic group-containing crosslinker component has been completely and/or homogenously dissolved within the resulting solution.

The ocular sealant formulations and/or dosage forms utilized in the present invention may in certain embodiments contain a surfactant. The surfactant may be a non-ionic surfactant. The non-ionic surfactant may comprise a poly(ethylene glycol) chain. Exemplary non-ionic surfactants are poly(ethylene glycol) sorbitan monolaurate commercially available as Tween® (and in particular Tween®20, a PEG-20-sorbitan monolaurate, or Tween®80, a PEG-80-sorbitan monolaurate), poly(ethylene glycol) ester of castor oil commercially available as Cremophor (and in particular Cremophor40, which is PEG-40-castor oil), and an ethoxylated 4-tert-octylphenol/formaldehyde condensation polymer which is commercially available as Tyloxapol and others such as Triton. A surfactant may aid in dispersing the components and may prevent particle aggregation, and may also reduce possible adhesion of the hydrogel strand to the tubing during drying. Other suitable surfactants for use in ocular sealant formulations and/or dosage forms of the present invetion include, but are not limited to, poly(ethylene glycol) sorbitan monolaurate, a polyethylene glycol-20-sorbitan monolaurate, a polyethylene glycol-80-sorbitan monolaurate), poly(ethylene glycol) ester of castor oil or an ethoxylated 4-tert-octylphenol/formaldehyde condensation polymer.

The ocular sealant formulations and/or dosage forms utilized in the present invention may in certain embodiments contain a solubility enhancer. The solubility enhancer may be present in an amount of about 1 wt. % to about 10 wt. %, about 2 wt. % to about 7 wt. %, or about 3 wt. % to about 5 wt. % based on the total dry weight of the ocular sealant formulation and/or dosage form.

Suitable solubility enhancers for use in ocular sealant formulations and/or dosage forms of the present invention include, but are not limited to a carrier, an effervescent, a disintegrant, a sorbitol isomer, a buffer, a co-solvent, a surfactant as described above, a lubricant, a binder, a filler, a flow aid or combinations of any of the foregoing. Suitable carriers include, but are not limited to, mannitol, D-mannitol, sucrose, maltose, lactose, inositol, dextran or any combination of the foregoing. Suitable sorbitol isomers include, but are not limited to, mannitol, D-mannitol, xylitol, sorbitol or any combination of the foregoing. In certain embodiments, ocular sealant formulations and/or dosage forms of the present invention include D-mannitol in an amount of about 1.0 wt. % to about 15 wt. %.

In certain embodiments, the ocular sealant formulations and/or dosage forms include an effervescent. Suitable effervescents for use in ocular sealant formulations and/or dosage forms of the present invention include, but are not limited to, sodium carbonate, sodium bicarbonate, adipic acid, malic acid, tartaric acid, ascorbic acid, fumaric acid, maleic acid, succinic acid, citric acid or any combination of the foregoing.

In certain embodiments, the ocular sealant formulations and/or dosage forms include a disintegrant. Suitable disintegrants include, but are not limited to, crospovidone, croscarmellose sodium, sodium starch glycolate, and/or combinations thereof.

In some embodiments, ocular sealant formulations and dosage forms as described herein may include a lubricant, flow aid and/or a binder. Suitable binders include, but are not limited to, povidone, hydroxypropyl cellulose, collagen, a polysaccharide, hyaluronan, a cellulose, carboxymethylcellulose, a polyol, polyvinyl alcohol, and/or combinations thereof. Suitable lubricants and/or flow aids include, but are not limited to, magnesium stearate, sodium stearyl fumarate, calcium stearate, stearic acid, glycerol behenate, polyethylene glycol, talc, mineral oil and/or combinations thereof.

In certain embodiments, the ocular sealant formulations and/or dosage forms include a buffer. Suitable buffers include, but are not limited to, citric acid, ascorbic acid, phosphoric acid, acetic acid, histidine lactic acid, tromethamine gluconic acid, aspartic acid, glutamic acid, tartaric acid, succinic acid, malic acid, fumaric acid, α-ketoglutaric acid, a citrate, a phosphate or an acetate. In certain particular embodiments, the buffer is contained in the solvent of the ocular sealant systems. The solvent may be water alone or saline or a combination of water or saline and the buffer.

In certain embodiments, the ocular sealant formulations and/or dosage forms include a co-solvent. Suitable co-solvents include, but are not limited to, dimethyl sulfoxide, ethanol, PEG 300, glycerol or benzyl alcohol.

In some embodiments, the ocular sealant formulations and/or doage forms include a filler. Suitable fillers include, but are not limited to, lactose, dextrose, mannitol, microcrystalline cellulose, or a mixture thereof.

In certain embodiments, the ocular sealant formulations and/or dosage forms include an active agent. Suitable active agents include, but are not limited to, a cyclodextrin, a steroid, an antibiotic, or a combination thereof. Suitable cyclodextrins include, but are not limited to, acetazolamide, anandamide, a cannabinoid, cyclosporin, a dehydroepiandrosterone, dexamethasone, diclofenac, dipivefrine, fluorometholone, hydrocortisone, loteprednol etabonate or pilocarpine. Suitable steroids include, but are not limited to, dexamethasone. Suitable antibiotics include, but are not limited to, azithromycin, bacitracin, neomycin, polymyxin B, mupirocin, nitrofurazone, fusidic acid or combinations thereof.

### Formulation:

In certain embodiments, ocular sealant formulations comprise a polymer network formed by combining a nucleophilic group-containing crosslinker component comprising less than about 500 ppm by weight water, an electrophilic group-containing multi-arm-polymer precursor component comprising less than about 1 wt. % water and a solvent comprising at least one salt.

In certain embodiments, ocular sealant formulations may be in the form of a solid dosage form, comprising a compressed mixture of a nucleophilic group-containing crosslinker component, an electrophilic group-containing multi-arm-polymer precursor component, and at least one excipient. Such ocular sealant dosage forms may comprise less than about 5 wt. % water.

In some embodiments, the ocular sealant formulations and/or dosage forms according to the present invention in a dry state contain from about 30% to about 95% by weight electrophilic group-containing multi-arm-polymer precursor component (e.g., units of polyethylene glycol 8a15kSS), or any value or sub-range within this range, and from about 1% to about 60% by weight nucleophilic group-containing crosslinker component (e.g., trilysine acetate, amine-terminated PEG such as 8a20kNH2) such as those disclosed above, or any value or sub-range within this range. In further embodiments, the ocular sealant formulations and/or dosage forms according to the present invention in a dry state contain from about 85% to about 95% by weight, or about 35% to about 60% by weight electrophilic group-containing multi-arm-polymer precursor component (e.g., units of polyethylene glycol 8a15kSS), and from about 1% to about 10% by weight, or about 40% to about 55% by weight nucleophilic group-containing crosslinker component (e.g., trilysine acetate, amine-terminated PEG such as 8a20kNH2), such as those disclosed above.

In certain other embodiments, the ocular sealant formulations and/or dosage forms according to the present invention in a dry state contain from about 90% to about 95% by weight, or about 49% to about 55% by weight electrophilic group-containing multi-arm-polymer precursor component (e.g., units of polyethylene glycol 8a15kSS), and from about 36% to about 46% by weight, or about 41% to about 52% by weight nucleophilic group-containing crosslinker component (e.g., trilysine acetate, amine-terminated PEG such as 8a20kNH2), such as those disclosed above.

In certain embodiments, the ocular sealant formulations and/or dosage forms may contain in a dry state about 0.05% to about 1% by weight, or any individual amount or sub-range within this range, of a visualization agent, such as FD&C Blue No. 1 (briliant blue) or methylene blue. Ocular sealant formulations according to the present invention may further contain a solvent having about 0.0% to about 5% by weight of one or more buffer salt(s) (separately or taken together). In certain embodiments, the ocular sealant formulations and/or dosage forms in a dry state may contain, e.g., from about 0.01% to about 2% by weight or from about 0.05% to about 0.5% by weight of a solubility enhancer.

The ocular sealant formulations and/or dosage forms utilized in the present invention may in certain embodiments contain a solubility enhancer (e.g., D-mannitol). The solubility enhancer may be present in an amount of about 1 wt. % to about 10 wt. %, about 2 wt. % to about 7 wt. %, or about 3 wt. % to about 5 wt. % based on the total dry weight of the ocular sealant formulation and/or dosage form.

Suitable solubility enhancers for use in ocular sealant formulations and/or dosage forms of the present invention include, but are not limited to a carrier, an effervescent, a sorbitol isomer, a buffer, a co-solvent, a surfactant as described above, or combinations of any of the foregoing. Suitable carriers include, but are not limited to, mannitol, D-mannitol, sucrose, maltose, lactose, inositol, dextran or any combination of the foregoing. Suitable sorbitol isomers include, but are not limited to, mannitol, D-mannitol, xylitol, sorbitol or any combination of the foregoing. In certain embodiments, ocular sealant formulations and/or dosage forms of the present invention include D-mannitol in an amount of about 1.0 wt. % to about 15 wt. %.

In certain embodiments, the ocular sealant formulations and/or dosage forms include an effervescent. Suitable effervescents for use in ocular sealant formulations and/or dosage forms of the present invention include, but are not limited to, sodium carbonate, sodium bicarbonate, adipic acid, malic acid, tartaric acid, ascorbic acid, fumaric acid, maleic acid, succinic acid, citric acid or any combination of the foregoing.

In certain embodiments, the ocular sealant formulations and/or dosage forms include a buffer. Suitable buffers include, but are not limited to, citric acid, ascorbic acid, phosphoric acid, acetic acid, histidine lactic acid, tromethamine gluconic acid, aspartic acid, glutamic acid, tartaric acid, succinic acid, malic acid, fumaric acid, α-ketoglutaric acid, a citrate, a phosphate or an acetate. In certain particular embodiments, the buffer is contained in the solvent of the ocular sealant systems. The solvent may be water alone, saline alone, or a combination of water or saline and the buffer.

In certain embodiments, the ocular sealant formulations and/or dosage forms include a co-solvent. Suitable co-solvents include, but are not limited to, dimethyl sulfoxide, ethanol, isopropyl alcohol, PEG 300, glycerol or benzyl alcohol.

In certain embodiments, the balance of the ocular sealant formulation and/or dosage form in its dry state, that is, the remainder of the formulation when the polymer hydrogel (i.e., nucleophilic group-containing crosslinker component and electrophilic group-containing multi-arm-polymer precursor component), visualization agent (e.g., FD&C Blue, methylene blue, etc.) and solubility enhancer have already been taken account of, may be salts remaining from the buffer in the solvent used to dissolve the other components and to form the ocular sealant formulation, or may be other ingredients used during manufacturing of the ocular sealant systems and dosage forms. In certain embodiments, such salts are phosphate, borate or (bi) carbonate salts. In one embodiment a buffer salt is sodium phosphate (mono- and/or dibasic).

The amount of the polymer(s) may be varied, and other amounts of the polymer hydrogel than those disclosed herein may also be used to prepare ocular sealant formulations and systems utilized in the invention. Similarly, in embodiments containing an active agent(s) (e.g., an antibiotic, a steroid), the amount of the active agent may be varied, and other amounts of the active agent other than those disclosed herein may also be used to prepare ocular sealant formulations and systems utilized in the invention.

In certain embodiments, the maximum amount (in weight %) of active agent within the formulation is about two times the amount of the polymer (e.g., PEG) units, but may be higher in certain cases, as long as the mixture comprising e.g., the precursors, visualization agent, buffers and drug (in the state before the hydrogel has gelled completely) can be uniformly applied to the surface of a subject's eye.

In certain embodiments, solid contents of about 20% to about 50% (w/v) (wherein “solids” means the combined weight of polymer precursor(s), optional visualization agent, salts and the drug in solution) are utilized for forming the hydrogel of the ocular sealant formulations according to the present invention.

In certain embodiments, the water content of the hydrogel in a dry (dehydrated/dried) state may be low, such as not more than about 1% by weight of water. The water content may in certain embodiments also be lower than that, possibly no more than about 0.25% by weight or even no more than about 0.1% by weight.

### Containers for Ocular Sealant Systems:

In certain embodiments, ocular sealant systems of the present invention include at least one container (e.g., a tray). The container may be configured to store the nucleophilic group-containing crosslinker component and the electrophilic group-containing multi-arm-polymer precursor component. In certain embodiments, the container is formed of a high-density polyethylene, a polycarbonate, a polystyrene, a nylon, an aluminum foil, polyethylene terephthalate (PET), polyvinyl chloride (PVC), polyvinylidene chloride (PVDC), polychlorotrifluoroethylene (PCTFE), cyclic olefin polymers (COP) and/or combinations thereof. The container may include less than about 0.50 wt. % water, less than about 0.25 wt. % water, less than about 0.20 wt. % water, less than about 0.15 wt. % water, or about 0.1 wt. % to about 0.5 wt. % based on the total weight of the container, or any individual amount or sub-range within these ranges. In certain embodiments, the container includes a water absorbance value of less than about 0.50% water, less than about 0.25% water, less than about 0.20% water, less than about 0.15% water, or about 0.1 wt. % to about 0.5 wt. % based on the total weight of the container, or any individual amount or sub-range within these ranges.

### Biodegradation of Ocular Sealant:

In one embodiment, the present invention relates to a biodegradable ocular sealant comprising a hydrogel. In some embodiments, the ocular sealant releases a therapeutically effective amount of an active agent (e.g., an antibiotic) as described herein. In certain embodiments, the ocular sealant provides an immediate release of the active agent, a sustained release of the active agent, or a mixed release. For example, the ocular sealant may release an active agent for at least about 1 day to about 30 days after administration (i.e., after the ocular sealant formulation is topically applied to the surface of an eye). In a particular embodiment, suitable antibiotics include, but are not limited to, azithromycin, bacitracin, neomycin, polymyxin B, mupirocin, nitrofurazone and fusidic acid.

Without being bound by any particular theory, release of the active agent (e.g., an antibiotic, a glucocorticoid) into the tear fluid is determined by the active agent's solubility in an aqueous environment. One particular glucocorticoid for use according to the present invention is dexamethasone. The solubility of dexamethasone has been determined to be very low in an aqueous medium (less than 100 μg/mL), such as the tear fluid. When administered, the dexamethasone is released from the ocular sealant primarily at its surface proximal to the tear fluid and thus proximal to the eye surface.

In certain embodiments, the active agent gradually gets dissolved and diffuses out of the hydrogel into the tear fluid. This happens primarily in a unidirectional manner, starting at the interface of the ocular sealant and the tear fluid at the proximal surface of the applied sealant. The “drug front” generally progresses in the opposite direction, i.e., away from the proximal surface until eventually the entire ocular sealant is depleted of active agent.

In certain embodiments, the ocular sealant formulation according to the present invention provides for the release of an active agent, such as an antibiotic or dexamethasone, for a period of about 6 hours or longer, such as for a period of about 12 hours or longer, or 1 week or longer, or 2 weeks, or one month or longer, or any individual period or sub-range within these ranges.

In certain embodiments, after administration, the levels of active agent released from the ocular sealant per day remain constant or essentially constant over a certain period of time (due to the limitation of release based on the active agent's solubility), such as for about 7 days, or for about 11 days, or for about 14 days, or any individual period or sub-range within these ranges. Then the amount of active agent released per day may decrease for another period of time (also referred to as “tapering”), such as for a period of about 7 additional days (or longer in certain embodiments) until all or substantially all of the active agent has been released and the “empty” hydrogel remains on the surface of the eye until it is fully degraded and/or until it is cleared (disposed/washed out) by the subject.

Concurrently with the drug diffusing out of the hydrogel (and also after the entire amount of drug has diffused out of the hydrogel), the hydrogel may be slowly degraded e.g. by means of ester hydrolysis in the aqueous environment of the tear fluid. At advanced stages of degradation, distortion and erosion of the hydrogel begins to occur. As this happens, the hydrogel becomes softer and more liquid (and thus its shape becomes distorted) until the hydrogel finally dissolves and is resorbed completely.

In one embodiment, the persistence of the hydrogel within an aqueous environment such as in the human eye depends inter alia on the structure of the linker that crosslinks the polymer units, such as the PEG units, in the hydrogel. In certain embodiments, the hydrogel is biodegraded within a period of about 1 month, or about 2 months, or about 3 months, or up to about 4 months after administration, or any individual value or sub-range within these ranges. However, since during the degradation process in the aqueous environment, such as in the tear fluid, the hydrogel gradually becomes softer and distorted, the ocular sealant may be cleared (washed out/disposed) before it is completely biodegraded.

In embodiments of the present invention, the hydrogel and thus the ocular sealant remains on the surface of the eye for a period of up to about 1 day, or up to about 2 days, or up to about 3 days, or up to about 4 days, or up to about 5 days, or up to about 1 week, or up to about 2 weeks, or about 1 day to about 30 days after administration, or any individual time or sub-range within these ranges.

In certain embodiments of the invention, the entire amount of active agent may be released prior to the complete degradation of the hydrogel, and the ocular sealant may persist on the surface of the eye thereafter, for a period of altogether up to up to about 1 day, or up to about 2 days, or up to about 3 days, or up to about 4 days, or up to about 5 days, or up to about 1 week, or up to about 2 weeks, or about 1 day to about 30 days after administration, or any individual time or sub-range within these ranges. In certain other embodiments, the hydrogel is fully biodegraded when the active agent has not yet been completely released from the ocular sealant.

In certain embodiments, in vitro release tests may be used to compare different ocular sealants (e.g. of different production batches, of different composition, and of different dosage strength etc.) with each other, for example for the purpose of quality control or other qualitative assessments. The in vitro-release of an active agent from the ocular sealant of the invention can be determined by various methods, such as under non-sink simulated physiological conditions in PBS (phosphate-buffered saline, pH 7.4) at 37° C., with daily replacement of PBS in a volume comparable to the tear fluid in the human eye.

### II. Manufacture of Systems and Dosage Forms

In certain embodiments, methods of preparing an ocular sealant system, comprises depositing a nucleophilic group-containing crosslinker component on a surface of the container, drying the nucleophilic group-containing crosslinker component and the container, depositing a molten electrophilic group-containing multi-arm-polymer precursor component on a surface of the container, and sealing the container having the nucleophilic group-containing crosslinker component and the electrophilic group-containing multi-arm-polymer precursor component thereon in a storage container.

In certain embodiments, methods of preparing an ocular sealant dosage form, comprise milling an electrophilic group-containing multi-arm-polymer precursor component, a nucleophilic group-containing crosslinker component and at least one excipient to form milled components and mixing the milled components to form a mixture. The mixture may then be compressed to form at least one tablet.

In certain embodiments the method of manufacturing the ocular sealant formulation to utilize in the present invention comprises the steps of forming a hydrogel comprising a polymer network (e.g., comprising PEG units) and glucocorticoid particles dispersed in the hydrogel, shaping or casting the hydrogel and drying the hydrogel. In one embodiment the glucocorticoid, such as dexamethasone, may be used in micronized form as disclosed herein for preparing the ocular sealant formulation. In another embodiment, the glucocorticoid, such as dexamethasone, may be used in non-micronized form for preparing the ocular sealant formulation.

Suitable precursors for forming the hydrogel of certain embodiments of the invention are as disclosed above in the section relating to the ocular sealant formulations and dosage forms. In certain specific embodiments, the hydrogel is made of a polymer network comprising crosslinked polyethylene glycol units as disclosed herein. The polyethylene glycol (PEG) units in particular embodiments are multi-arm, such as 8-arm, PEG units having an average molecular weight from about 2,000 to about 100,000 Daltons, or from about 10,000 to about 60,000 Daltons, or from about 15,000 to about 50,000 Daltons, or of about 20,000 Daltons. Suitable PEG precursors having reactive groups such as electrophilic groups as disclosed herein are crosslinked to form the polymer network. Crosslinking may be performed by means of a crosslinking agent that is either a low molecular compound or another polymeric compound, including another PEG precursor, having reactive groups such as nucleophilic groups as also disclosed herein. In certain embodiments, a PEG precursor with electrophilic end groups is reacted with a crosslinking agent (a low-molecular compound, or another PEG precursor) with nucleophilic end groups to form the polymer network.

In certain particular embodiments, a method of manufacturing an ocular sealant system includes preparing a container (e.g., a mixing tray 108, 208, 408 as shown in FIGS. 1, 2A, 4A) for deposition, depositing a nucleophilic group-containing crosslinker component, depositing an electrophilic group-containing multi-arm-polymer precursor component, preparing a diluent solution and packaging the components and diluent together in a storage container. Preparing the container may include adding water and soap to a large volume beaker. A sufficient number of polycarbonate trays may be added to the soap and water solution. In certain embodiments, a probe sonicator may be submerged in the soap and water solution. The containers may be cleaned using a brush. Once removed from the soap and water, the containers may be rinsed with water for injection (WFI). The cleaned and rinsed trays may be placed in an incubator to dry at 37° C. for at least 12 hours.

In certain embodiments depositing the nucleophilic group-containing crosslinker component (e.g., trilysine acetate, amine-terminated polyethylene glycol) on the clean and dry tray may include weighing appropriate amounts of the nucleophilic group-containing crosslinker component (e.g., trilysine acetate, amine-terminated polyethylene glycol) and, optionally, a visualization agent (e.g., FD&C Blue #1). These components may be placed in a mixing vessel (e.g., a bottle) and subjected to a vortex for about 1 second to about 24 hours, about 5 seconds to about 12 hours, about 30 seconds to about 6 hours, or about 1 minute until about 3 hours, or any individual time or sub-range within these ranges, and/or until complete dissolution is observed. In some embodiments, a pH meter can be used to adjust the resulting mixture to a target pH, for example, an acidic pH, a basic pH, a pH of about 3 to about 10, about 4 to about 6, about 7 to about 9, or any individual value or sub-range within these ranges. The cleaned and dried trays may be placed in a securing device (e.g., in a mixing tray nest). A desired volume of the nucleophilic group-containing crosslinker component (e.g., in a flowable form) may be deposited (e.g., dropwise) onto the cleaned and dried container. In some embodiments, the nucleophilic component may be deposited using a pipette and/or a pipetting. In certain embodiments, the nucleophilic compnent may be deposited in an amount of about about 0.1 μL to about 100 μL, about 0.5 μL to about 50 μL, or about 1 μL to about 25 μL, or any individual value or sub-range within these ranges. In certain particular embodiments, the nucleophilic component may be deposited in an amount of about less than 30 μL, about 1 μL to about 29 μL, about 2 μL to about 25 μL, or about 5 μL to about 20 μL, or any individual volume or sub-range within these ranges, of the nucleophilic component (e.g., TLA, amine-terminated PEG) is deposited onto the basin 114a, 114b, 214a, 214b, 414a, 414b of the container 108a, 108b, 208a, 208b, 408a, 408b.

Once the nucleophilic component has been deposited onto the basins of the containers seated in the nest, the containers may be placed in a vessel (e.g., a stoppering tote) and isolated (e.g., sealed with rubber stoppers placed into nest openings). The containers having the nucleophilic component deposited thereon may then be allowed to dry at room temperature (e.g., about 20° C. to about 25° C.) or baked in a vacuum oven. The vacuum oven may be preheated to a temperature of about 45° C. to about 80° C., about 55° C. to about 75° C., about 60° C. to about 70° C., or any individual temperature or sub-range within these ranges. In some embodiments, nitrogen gas may be introduced into the vacuum chamber at a flow rate of about 20 SCFH to about 60 SCFH, 30 SCFH to about 40 SCFH, or any individual value or sub-range within these ranges. The tote having the mixing trays with the deposited nucleophilic component thereon may be sealed in the vacuum chamber and heated for about 30 minutes to about 3 hours, about 1 hour to about 2 hours, or any individual time or sub-range within these ranges. After baking, the nitrogen gas flow may be turned off, and the vacuum pump may be turned on. The mixing trays with the nucleophilic component thereon may continue heating for an additional about 15 minutes to about 2 hours, about 30 minutes to about 1 hour, or any individual time or sub-range within these ranges. Following this drying cycle, the vacuum chamber door may be opened and the stoppered tote removed. The tote may be transferred to a glove box and allowed to cool.

In certain embodiments, rather than depositing the nucleophilic component (e.g., trilysine acetate, amine-terminated polyethylene glycol, etc.) on the basin of the container, the nucleophilic component may be deposited on an end of applicator 417 as shown and described with respect to FIGS. 4A-4C. The method may include applying the nucleophilic component as a drop onto a tip end of the applicator 417 as shown in FIG. 4B. Allowing the drop 402a to spread forms a thin film of the nucleophilic component 402b on the applicator 417.

In some embodiments, the method includes heating drop 402a on applicator 417 to cause the drop 402a to spread and form thin film 402b. In certain embodiments, the method further includes baking and drying the thin film of the nucleophilic component 402b onto the tip end of applicator 402b. Applicator 417 having the nucleophilic component thereon may then be sealed in a container (e.g., an inert plastic bag, a vial, etc.) (not shown). Because of the reduced volume, the concentration of the nucleophilic group-containing crosslinker component in thin film 402b may be higher than in a nucleophilic component deposited onto basin 414a, 414b. Depositing the nucleophilic component 402b on applicator 417 removes water from the container 408 manufacturing process as the nucleophilic component typically requires pre-dissolution of the component in water prior to deposition onto container 408. Additionally, separately packaging the applicator having the nucleophilic component thereon isolates the electrophilic component 403a, 403b from possible water contamination thus increasing the shelf-life of the components within system 400.

Ocular sealant system 400 further includes a solvent bottle 414, as described hereinabove, which holds and stores a solvent 416. Container 408, like containers 108, 208, has an electrophilic group-containing multi-arm-polymer precursor component 403a, 403b printed on an elongate portion 409a, 409b of recess 410a, 410b. The electrophilic component 403a, 403b may be printed in a pattern, e.g., small lines or dots, as illustrated by the ladder pattern in FIG. 4A. Solvent 416 may be added dropwise (or pipetted) into recess 410a, 410b whereupon contact, the electrophilic component 403a, 403b will begin to dissolve in the solvent 416. The tip end of applicator 417 having the thin film of nucleophilic component 402B deposited thereon may be submerged in the solvent 416 and electrophilic component 403a, 403b mixture. Applicator 417 may be used to mix the formulation within recess 410a, 410b and while mixing, the nucleophilic component 402b dissolves into the solution.

In certain embodiments, the method of manufacturing an ocular sealant system may further include depositing the electrophilic group-containing multi-arm-polymer precursor component (e.g., 8a15kSS). The electrophilic component may be deposited via a printing apparatus onto a container having the nucleophilic component deposited and baked thereon as described above. The printing apparatus may be pre-heated to a temperature of about 45° C. to about 100° C., about 50° C. to about 90° C., about 60° C. to about 85° C., or any individual temperature or sub-range within these ranges. In some embodiments, the system settings on the print head controller may be verified and the barrel and nozzle temperatures heated to the aforementioned pre-heating temperatures. The waveform and/or the amplitude may be optimized to provide the desired patterning and control of the deposition. For example, if the waveform is too aggressive, splattering of the polymer can occur. The amplitude may be optimized as well to control the patterning (e.g., depositing clean lines and/or dots). In some embodiments, the waveform (ms), which controls how the piston behaves when dispensing the molten PEG, may be set to T1=about 0.100 to about 0.500 or about 0.200 to about 0.400, T2=about 0.050 to about 0.400, or about 0.100 to about 0.300 and T3=about 1.0 to about 5.0, or about 2.0 to about 3.1, or any individual values or sub-ranges within these ranges. The amplitude of the printing apparatus may be about 50% to about 100%, about 60% to about 90%, about 76% to about 85%, or any individual amplitude or sub-range within these ranges. When the printing apparatus reaches the desired temperature and all settings have been entered and verified (e.g., that the O₂ and moisture levels are less than about 40 ppm by weight, less than about 30 ppm by weight, less than about 20 ppm by weight, or less than about 10 ppm by weight, or any individual value or sub-range within these ranges, in the glove box) the three-axis robot may begin its deposition process.

The electrophilic component (e.g., with 8a15kSS PEG) may be added to a melting reservoir of the printing apparatus. The electrophilic component may be heated at a temperature of about 45° C. to about 100° C., about 50° C. to about 90° C., about 60° C. to about 85° C., or any individual temperature or sub-range within these ranges, for at least about 5 min, at least about 10 min, at least about 15 min, at least about 20 min, or any individual duration or sub-range within these ranges, before the printing process begins. In certain embodiments, the mixing tray nest may be placed and secured onto the printing stage. The printing program may be initiated to dispense the molten electrophilic component onto the elongate portion of the recess of a container in a predetermined pattern (e.g., a ladder, dots, etc.) on the narrow end of the mixing trays (see, e.g., FIGS. 1A, 2A). The mixing trays having the deposited electrophilic component thereon may be removed from the printing stage and placed in an isolation chamber (e.g., a glove box, inert chamber). One or more tray may be sealed in an outer container (e.g., sealed in pouches) and/or packaging. For example, each mixing tray may be removed from the nest and placed into an outer package (e.g., a foil pouch such as a DESSIFLEX® pouch), which is then sealed. In some embodiments, the packaged trays may be further packaged together with other components to form an ocular sealant system (e.g., as a system).

In certain embodiments, a solvent 116 may be prepared, stored in a container 214 and packaged with the packaged trays discussed above to form an ocular sealant system. The solvent may be prepared by weighing appropriate amounts of one or more salts (e.g., sodium phosphate dibasic, sodium phosphate monobasic and sodium borate decahydrate). In certain embodiments, the solvent includes a monobasic salt and a dibasic salt. The monobasic salt and dibasic salt may be present at a weight ratio of the monobasic to the dibasic of about 1:10 to about 10:1, about 1:8 to about 8:1, about 1:5 to about 5:1, or any individual ratio combination or sub-range within these ranges. In some embodiments, the salts include sodium phosphate dibasic, sodium phosphate monobasic and sodium borate decahydrate, which may be present in the solvent at a weight ratio of about 1:1:1 to about 10:1:1, or 1:1:1 to about 1:10:1, or about 1:1:1 to about 1:1:10, or any individual ratio combination or sub-range within these ranges. WFI may be added to a mixing vessel together with the one or more salts. The solution may be mixed for about 1 second to about 30 minutes, about 5 seconds to about 20 minutes, about 15 seconds to about 15 minutes, or any individual duration or sub-range within these ranges, until the one or more salts are completely dissolved within the WFI. The resulting solvent may be poured into a solvent container (e.g., a dropper bottle, a pipette, etc.) as described herein and sealed.

In certain embodiments, to prepare an ocular sealant system as described herein, at least one packaged mixing tray having the nucleophilic and electrophilic components deposited thereon may be packaged together with a solvent container, for example, in a TYVEK® pouch. In some embodiments, one or more applicators 117, 417 may be also be packaged with the mixing tray and solvent container to form the ocular sealant system.

An embodiment of a method 700 of manufacturing an ocular sealant dosage form is shown in FIG. 7. In certain embodiments, to reduce or prevent contamination, the method of forming the ocular sealant dosage form may be completed within an inert environment (e.g., a glove box, an inert vessel, etc.). Method 700 may include, at block 702, forming particles comprising an electrophilic group-containing multi-arm-polymer precursor component, a nucleophilic group-containing crosslinker component and at least one excipient. The term “particles” as used herein refers to any sub-unit of a larger mass and includes, but is not limited to, powder, extrudates, granules, beads, pellets, milled particles, substrates, spheres, microspheres, multi-particulates (e.g., a particle formed of more than one component), nanoparticles and any combination of two or more of the foregoing. In some embodiments, the particles may be coated with one or more of an inert coating, a film coating, a cosmetic coating or an active agent containing coating configured to provide an immediate release and/or a sustained release of the active agent. Forming particles 702 may be performed independently for each of the electrophilic group-containing multi-arm-polymer precursor component, the nucleophilic group-containing crosslinker component, and the at least one excipient. For example, forming particles 702 may include separately milling, grinding, extruding, granulating, etc. the electrophilic group-containing multi-arm-polymer precursor component, the nucleophilic group-containing crosslinker component and the at least one excipient. In certain particular environments, milling may be performed in the presence of dry ice. For example, dry ice and the component to be milled may be placed in a grinding chamber. The component is milled in the presence of dry ice to maintain a cool temperature while milling. After milling, the dry ice is allowed to sublimate, after which the components can be used for processing. Each of the components in particular form may be transferred to a storage container (e.g., a labeled vial) to hold for future use.

Method 700 may further include at block 704 combining the particles to form a combination. Combining the particles 704 may include weighing particles of each component to obtain a desired amount of each individual component. The electrophilic group-containing multi-arm-polymer precursor component and the nucleophilic group-containing crosslinker component may be combined at a weight ratio of the electrophilic component to the nucleophilic component of about 1:50 to about 50:1, about 1:40 to about 40:1, about 1:30 to about 30:1, about 1:20 to about 20:1, about 1:17 to about 17:1, about 1:15 to about 15:1, about 1:10 to about 10:1, about 1:5 to about 5:1, about 1:1.5 to about 1.5:1, about 0.75:1 to about 1:0.75, or any individual ratio or sub-range within these ranges. Each of the weighed components may mixed (e.g., in a mixing vessel). The components may be mixed for about 1 second to about 45 minutes, or any individual value or sub-range within this range, to sufficiently mix the components. For example, the mixing vessel (e.g., a vial) may be placed in a tumble mixer such that the mixing vessel is as tumbled end-to-end at a rate of about 5 rpm for about 25 rpm until the components are completely mixed.

Method 700 may further include at block 706, compressing the combination of mixed particles to form at least one tablet or a solid applicator (e.g., a crayon, a cylinder, etc.). For example, a tablet press may be used to compress a pre-determined mass of the combination to form a tablet or solid applicator having the desired amounts of the components at the established ratios. In certain embodiments, the tablet press may compress the combination at a force of about 0.3 lbf to about 1.2 lbf. The resulting tablet or solid applicator may include at least one excipient that causes effervescence and/or dissolution (e.g., homogenous dissolution) when the tablet is contacted with a solvent. In certain particular embodiments, the resulting tablet or solid applicator may be stored in a mixing tray, for example, as shown in FIG. 5 and sealed by an outer package. The sealed mixing tray and tablet or solid applicator may be further packaged with a solvent stored within a container (e.g., a dropper bottle) 514. For systems containing a tablet, at least one applicator 517 may be further packaged with the mixing tray and solvent to form the ocular sealant system.

In certain embodiments, the method of manufacturing the ocular sealant dosage form of the present invention comprises mixing and reacting an electrophilic group-containing multi-arm polyethylene glycol, such as 8a15kSS, with a nucleophilic group-containing crosslinking agent, such as trilysine acetate or an amine-terminated PEG, in a buffered solution optionally in the presence of active agent particles, and allowing the mixture to gel. In certain embodiments, the molar ratio of the electrophilic groups in the PEG precursor to the nucleophilic groups in the crosslinking agent is about 1:1, but may also be in a range from about 2:1 to about 1:2.

In certain embodiments, a visualization agent as disclosed herein is included in the nucleophilic component so that dissolution of this component can be visualized once it has been contacted with the solvent. For example, the visualization agent may be a colorant such as FD&C Blue #1.

In certain particular embodiments, during the manufacture of an ocular sealant formulation of the present invention an mixture/suspension of an active agent and the PEG precursor(s), such as an antibiotic and the 8a15kSS, and optionally an excipient (e.g., a solubility enhancer) may be prepared, deposited and baked onto a mixing tray. This antibiotic/PEG precursor mixture may then be combined with the solvent, crosslinking agent and optional visualization agent. The resulting ocular sealant solution thus contains the antibiotic, the polymer precursor(s), the crosslinking agent, the visualization agent and the one or more buffer salts.

In certain embodiments, once the ocular sealant formulation of the electrophilic group-containing polymer precursor, the nucleophilic group-containing crosslinking agent, the active agent, such as an antibiotic, optionally the visualization agent, and the buffered solvent has been prepared (i.e., after these components have been combined), the resulting mixture may be topically applied to the surface of an eye. The formulation may be spread over a scratch or other wound.

An embodiment of manufacturing an ocular sealant film can include forming particles comprising an electrophilic group-containing multi-arm-polymer precursor component, a nucleophilic group-containing crosslinker component and at least one excipient (e.g., a colorant, mannitol, etc.). In some embodiments, the particles may be coated with one or more of an inert coating, a film coating, a cosmetic coating or an active agent containing coating configured to provide an immediate release and/or a sustained release of the active agent. In some embodiments, forming particles may be performed independently for each of the electrophilic group-containing multi-arm-polymer precursor component, the nucleophilic group-containing crosslinker component, and the at least one excipient. For example, forming particles may include separately milling, grinding, extruding, granulating, etc. the electrophilic group-containing multi-arm-polymer precursor component, the nucleophilic group-containing crosslinker component and the at least one excipient. In certain particular environments, milling may be performed in the presence of dry ice. For example, dry ice and the component to be milled may be placed in a grinding chamber. The component is milled in the presence of dry ice to maintain a cool temperature while milling. After milling, the dry ice is allowed to sublimate, after which the components can be used for processing. Each of the components in particular form may be transferred to a storage container (e.g., a labeled vial) to hold for future use.

The method of manufacturing a film may further include combining the particles to form a combination. Combining the particles may include weighing particles of each component to obtain a desired amount of each individual component. The electrophilic group-containing multi-arm-polymer precursor component and the nucleophilic group-containing crosslinker component may be combined at a weight ratio of the electrophilic component to the nucleophilic component of about 1:50 to about 50:1, about 1:40 to about 40:1, about 1:30 to about 30:1, about 1:20 to about 20:1, about 1:17 to about 17:1, about 1:15 to about 15:1, about 1:10 to about 10:1, about 1:5 to about 5:1, about 1:1.5 to about 1.5:1, about 0.75:1 to about 1:0.75, or any individual ratio or sub-range within these ranges. Each of the weighed components may be mixed (e.g., in a mixing vessel). The components may be mixed for about 1 second to about 45 minutes, or any individual value or sub-range within this range, to sufficiently mix the components. For example, the mixing vessel (e.g., a vial) may be placed in a tumble mixer such that the mixing vessel is as tumbled end-to-end at a rate of about 5 rpm for about 25 rpm until the components are completely mixed. In some embodiments, the particles may be mixed in a dual asymmetric centrifuge (DAC). The components may be added to a reservoir of the mixer together with ceramic beads that are optionally heated. In certain embodiments, the combination is homogenous.

The method of forming the film may further include, extruding the combination of mixed particles to form a thin film. In some embodiments, the extrusion process is a melt extrusion process such that the combinaton of mixed particles are at least partially heated and/or melted prior to extrusion. The extruded film may be crushed or broken to form particles, mixed again and then extruded again. This process can be repeated as needed to result in a homogenous mixture within the extruded film. The thickness of the extruded film may be about 0.1 mm to about 5 mm, or any indvidual thickness or sub-range within this range.

In some embodiments, the extruded film may be stamped in any suitable shape (e.g., square, rectangle, circle, oval, etc.). To provide a suitable thickness, softness, flexibility and/or rigidity, the ratio of the nucleophilic component to the electrophilic component may be varied. In some embodiments, a weight ratio of the nucleophilic component to the electrophilic component may be about 20:1 to about 1:20, about 1:1 to about 1:20, about 20:1 to about 1:10, or any individual ratio or sub-range within this range. In some embodiments, the molecular weight of the nucleophilic component and/or the electrophilic component may be varied to provide a desired softness or hardness. The modulus of the hydrogel can be adjusted by the number of arms within the nucleophilic component and/or the electrophilic component. The volume of the combination of mixed particles, alone or in combination with the extrusion force, also may be adjusted to control thickness and/or hardness of the film.

In some embodiments, the combination of mixed particles may be extruded into a different shape, such as a rod. At least a portion of the rod may be further extruded into a film. In certain embodiments, the combination of mixed particles may be compressed and then supplied to the extrusion apparatus.

### III. Therapy

In one embodiment, the present invention relates to a method of treating a wound on an eye of a patient in need thereof, the method comprising administering to the patient's eye an ocular sealant formulation as disclosed herein. The patient may be a human or animal subject in need of such therapy. In certain embodiments, the treatment is for a scratch, incision and/or wound on the surface of the patient's eye.

In one embodiment, the present invention relates to a method of treating a wound on an eye of a patient in need thereof, the method comprising administering to the patient a biodegradable ocular sealant formulation containing an active agent as disclosed herein. In certain particular embodiments, the active agent is an antibiotic to prevent infection or dexamethasone to treat allergic conjunctivitis, prevent ocular itching and/or prevent redness.

In one embodiment, the ocular sealant formulation described herein releases a total of about 0.1 mg to about 10 mg, about 0.2 mg to about 8 mg, about 0.5 mg to about 5 mg, or about 1.0 mg to about 10 mg of antibiotic following administration. Suitable antibiotics include, but are not limited to, azithromycin, bacitracin, neomycin, polymyxin B, mupirocin, nitrofurazone and fusidic acid. In certain embodiments, following administration, the ocular sealant may provide a sustained release of the antibiotic.

In one embodiment, the ocular sealant formulation described herein releases at least 0.4 mg of dexamethasone following administration. Alternatively, as part of another embodiment, the ocular sealant formulation described herein releases at most 0.4 mg of dexamethasone following administration. In another alterative embodiment, the ocular sealant formulation described herein releases about 0.4 mg of dexamethasone following administration. In another embodiment, the ocular sealant formulation described herein releases about 0.4 mg of dexamethasone for up to 30 days following administration.

In another embodiment, treating as disclosed herein comprises a reduction in ocular itching following administration, e.g., of at least 15-days following administration.

In another embodiment, the allergic conjunctivitis as disclosed herein is caused by allergens selected from seasonal allergens and perennial allergens. Alternatively, as part of a another embodiment, the allergic conjunctivitis as disclosed herein is caused by allergens selected from timothy grass, white birch, meadow fescue, ragweed, Kentucky bluegrass, rye grass, maple, oak, dust mites, cat dander, cockroach, and dog dander.

In certain embodiments, the ocular sealant formulation remains one the eye after complete depletion of the active agent until the hydrogel has biodegraded and/or is disposed (washed out/cleared). As the hydrogel matrix of the ocular sealant formulation is formulated to biodegrade e.g. via ester hydrolysis in the aqueous environment of the tear fluid in the canaliculus, the ocular sealant formulation softens and liquefies over time. However, in case the ocular sealant should be removed e.g. because of a potential allergic reaction or other circumstances which require removal, such as an unpleasant foreign body sensation felt by a patient, or because treatment should be terminated for another reason, the ocular sealant may be manually removed.

In certain embodiments, the ocular sealant remains on the eye for up to about 1 day, about 2 days, about 3 days, about 4 days, about 5 days, about 6 days, about 1 week, about 2 weeks, about 3 weeks, about 1 month, or up to about 2 months after administration.

In certain embodiments the systemic concentration of glucocorticoid such as dexamethasone after administration of the ocular sealant formulation of the present invention is very low, such as below quantifiable amounts. This significantly reduces the risk of drug-to-drug interactions or systemic toxicity, which can be beneficial e.g. in older patients who are frequently suffering from ocular diseases and are additionally taking other medications.

### IV. Ocular Sealant Systems

In certain embodiments, the present invention is further directed to a system comprising one or more components of an ocular sealant formulation as disclosed herein or manufactured in accordance with the methods as disclosed herein.

In certain specific embodiments, the system comprises one or more sustained release biodegradable ocular sealants, wherein each dosage of ocular sealant contains from about 160 μg to about 250 μg or from about 180 μg to about 220 μg or about 200 μg dexamethasone and has in a dry state an average diameter in the range of about 0.41 mm to about 0.49 mm and an average length in the range of about 2.14 mm to about 2.36 mm, and has in the hydrated state an average diameter in the range of about 1.35 mm to about 1.80 mm and a ratio of length to diameter of greater than 1, and wherein each ocular sealant dosage provides for a release of dexamethasone for a period of up to about 14 days after administration.

In certain other specific embodiments, the system comprises one or more sustained release biodegradable ocular sealant formulations, wherein each dosage of ocular sealant contains from about 240 μg to about 375 μg or from about 270 μg to about 330 μg or about 300 μg dexamethasone and has in a dry state an average diameter in the range of about 0.44 mm to about 0.55 mm and an average length in the range of about 2.14 mm to about 2.36 mm, and has in the hydrated state an average diameter in the range of about 1.35 mm to about 1.80 mm and a ratio of length to diameter of greater than 1, and wherein each dosage of ocular sealant provides for a release of dexamethasone for a period of up to about 21 days after administration.

In certain embodiments, the system further comprises instructions for using the one or more sustained release biodegradable ocular sealant formulations. The instructions for mixing and applying the one or more iodegradable ocular sealant formulations may be in the form of an operation manual for the physician who is administering the ocular sealant formulation. The system may further comprise a package insert with product-related information.

In certain embodiments, the system may further comprise one or more means for administration of the one or more biodegradable ocular sealant formulations. The means for administration may be for example one or more suitable applicator, foam, brush, applicator solid or a combination thereof any one of which may be for single use or repeat use. The means for administration may also be a syringe, pipette, dropper or applicator system.

In certain embodiments, the components of the ocular sealant formulation together with one or more applicator may be packaged together for a single administration. In certain embodiments, one or more components of the ocular sealant formulation may be individually packaged for a single administration.

### EXAMPLES

The following Examples are included to demonstrate certain aspects and embodiments of the invention as described in the claims. It should be appreciated by those of skill in the art, however, that the following description is illustrative only and should not be taken in any way as a restriction of the invention.

### Example 1

### Preparation of an Ocular Sealant System Utilizing a Scalable Printing Platform

In this example, an ocular sealant system was prepared by melt depositing an electrophilic group-containing multi-arm-polymer precursor component (i.e., units of polyethylene glycol) in, for example, a ladder line geometry, as shown in FIGS. 1, 2A and 4A. In comparison, prior art methods for depositing a PEG electrophilic component involved lyophilizing a large aqueous mass of the PEG to form a lyophilized cake as shown in FIG. 3A. In this example, and in accordance with the methods of the invention, small amounts of melted, non-aqueous PEG was deposited onto a container using a printer. Depositing small amounts of melted, non-aqueous PEG using a printer substantially increases the deposition speed of this component as compared to the prior art lyophilization method. Dividing the PEG into a pattern of small amounts of material also provides for efficient dissolution of the electrophilic component upon contact with a solvent and aids in homogenously mixing the PEG component with the nucleophilic group-containing crosslinker component (i.e., trilysine acetate).

The printing method according to this example does not require pre-dissolution of the PEG component in water, as in the lyophilization technique, which comparatively reduces the moisture content by about 10×. As such, the printing method increases the shelf-life stability of the components within the ocular sealant system in comparison to the lyophilization deposition method. The increase in shelf life also may be attributed to a reduction in surface area of the printed PEG ladder pattern as compared to the porous lyophilized cake, wherein each line of the ladder has a half cylinder shape. A further benefit of the printing method employed in this example as compared to the lyophilization method is scalability. The printing method can be readily scaled up to increase production output of ocular sealant systems according to the invention.

Table 1 presents the components used to prepare the ocular sealant system according to this example.

TABLE 1
Components of the Ocular Sealant System

| | | Amount/System |
|---|---|---|
| Component | Amount/Well (mg) | (mg) |
| 8ARM 15k PEG - Succinimidyl Succinate (8a15kSS) | 8.28 | 16.56 |
| Trilysine Acetate (TLA) | 0.51 | 1.02 |
| FD&C Blue #1 | 0.02 | 0.04 |
| Sodium Phosphate Dibasic | 0.60 | 1.20 |
| Sodium Phosphate Monobasic | 0.04 | 0.08 |
| Sodium Borate Decahydrate | 0.35 | 0.70 |

### Process:

### Tray Preparation

1. Add water and soap to a large volume beaker.
2. Obtain necessary quantity of polycarbonate mixing trays (i.e., containers).
3. Add polycarbonate trays to soap and water solution.
4. Submerge a probe sonicator in the soap and water solution and leave submerged.
5. Use a brush to assist in cleaning the trays.
6. Remove trays from soap and water and rinse with water for injection.
7. Place trays into a 37° C. incubator to dry for at least 12 hours.

### Nucleophilic Component Deposit

1. Weigh the appropriate amounts of TLA and FD&C Blue #1 and place into a mixing bottle.
2. Vortex solution until complete dissolution is observed.
3. Use a pH meter to adjust solution to target. (Not sure if this should be included)
4. Obtain dried mixing trays and secure them in a mixing tray nest.
5. Get a deionizer and position over the trays prior to depositing solution.
6. Use a pipette to dispense TLA solution (20 μL—drop reduced from 30 μL aka 1.5× TLA formulation) onto both dimpled areas (concave indentations?) of the mixing tray.
7. Place nest into a stoppering tote, seal the tote, and place rubber stoppers into nest openings.
8. Preheat the vacuum oven to 60° C. and start N₂ (g) flow (40 SCFH) into the heating chamber.
9. Open vacuum oven door and place the tote with the mixing trays containing the TLA/FD&C Blue #1 solution into the oven chamber for 2 hours.
10. After 2 hours, turn off the N₂ (g) flow, and turn on the vacuum pump.
11. Continue heating for 1 hour.
12. After heating cycle is complete, open vacuum oven chamber door and stopper the tote.
13. Transfer sealed tote into a glove box and allow to cool before starting the PEG printing process.

### Melt Printer Setup

1. Turn on the printing apparatus to pre-heat the system and verify system settings on the print head controller.
2. Heat (° C.)
3. Barrel Temperature=85
4. Nozzle Temperature=85
5. Waveform (ms) (I could go into a bit of detail on these if needed, it's basically how the piston behaves to dispense the molten PEG)
   T1=0.200
   T2=0.300
   T3=3.1
6. Amplitude=76%
7. Turn on 3-axis robot stage and verify the correct printing pattern and settings are loaded.

### Electrophilic Component Deposit

1. Before printing verify that the O₂ and moisture levels are <20 ppm by weight in the glove box. (Not sure if this is necessary, just our internal specs)
2. Fill syringe with 8a15kSS PEG and place into melt printer.
3. Allow PEG to melt for at least 10 min before printing begins.
4. Place and secure the mixing tray nest onto the printing stage.
5. Initiate the printing program to dispense the molten PEG into the predetermined line pattern on the narrow end of the mixing trays.
6. Upon completion of the print program, remove nest from printing stage and set aside until ready to seal in pouches.
7. Remove mixing trays from nest and place one tray into a foil pouch (not sure if type of pouch is necessary to state—Dessiflex pouch) and seal.
8. Removed sealed pouches from the glove box and set aside until ready for final system packaging.

### Diluent Solution

1. Weigh the appropriate amounts of sodium phosphate dibasic, sodium phosphate monobasic, and sodium borate decahydrate.
2. Setup a beaker with WFI with a mixing bar on a stir plate.
3. Add salts to the WFI and stir until fully dissolved.
4. Obtain dropper doser, dropper tip, and dropper bottle.
5. Fill diluent dropper with appropriate volume of diluent solution.
6. Complete dropper assembly.

### System Packaging

1. Obtain dropper bottle, applicator assembly, and sealed mixing tray pouch and place into Tyvek pouch.
2. Seal Tyvek Pouch to complete assembly.

### Example 2

### Preparation of an Ocular Sealant Dosage Form

An effervescent tablet was prepared having the ingredients set forth in Table 2.

TABLE 2
Components of the Ocular Sealant Dosage Form

| Component | Per Tablet (mg) | Percent Composition |
|---|---|---|
| 8ARM 15k PEG - Succinimidyl Succinate (8a15kSS) | 4.28 | 21.4 |
| 8ARM 20k PEG - Amine (8a20kNH₂) | 5.70 | 28.5 |
| FD&C Blue #1 | 0.04 | 0.2 |
| Citric Acid | 4.54 | 22.7 |
| Sodium Bicarbonate | 5.44 | 27.2 |
| Total | 20.00 | 100% |

### Milling

1. All of the raw components listed in Table 2 were placed in a glove box. The entire process was completed in the glove box.
2. Each component was independently milled. Dry ice and the component to be milled were placed into a grinding chamber. The component is milled in the presence of dry ice to maintain a cool temperature while milling. After milling, the dry ice is allowed to sublimate, after which the components can be used for processing.
3. Each milled component was transferred into a labeled vial.

### Mixing

1. The desired weight of each milled component as set forth in Table 2 was measured by a scale. Each of the weighed components was combined in a mixing vessel (i.e., a mixing vial).
2. The mixing vessel was sealed and placed in a tumble mixer. The vial was tumbled end-to-end at a rate of about 5 rpm for about 25 rpm to fully mix the milled components.

### Pressing

1. The resulting mixture of the milled components was compressed using a tablet press. The tablet press was set to compress the mixture at a force of about 0.3 lbf to about 1.2 lbf.
2. The target amount of the mixture was weighed out on a scale and placed into the tablet press.
3. The lever was actuated to compress the milled mixture into an effervescent tablet.
4. The tablet was added to a mixing tray and sealed within a pouch.

### Example 3

### Comparison of Lyophilization vs. Drying Methods for Nucleophilic Component

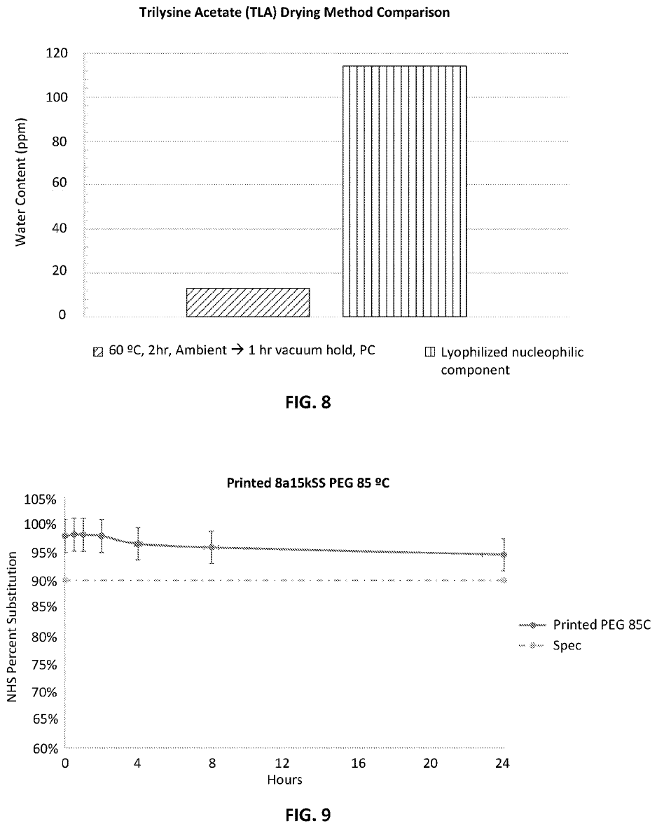

A mixture of trilysine acetate (TLA) and water was prepared and deposited onto a substrate and dried using the lyophilization technique in accordance with the prior art. The volume of the deposit was 30 μL in accordance with a prior art ocular sealant system. Separately, another mixture of TLA and water was deposited onto another substrate and was dried in a vacuum chamber for two hours at atmospheric pressure and a temperature of 60° C. After two hours, the pressure within the vacuum chamber was reduced to 200 mTorr and the nucleophilic component continued to dry for one more hour. The volume of this deposit, in accordance with embodiments of the invention, was about 20 μL. The water content of each deposited and dried nucelophilic component was measured. A Karl Fischer Moisture Analysis was performed on each deposited nucleophilic component dried by the two different methods. The KARL FISCHER analysis used to measure the water content may be a melting cure generation method (or loss on drying method) that includes ramping the temperature of a KARL FISCHER oven from 50° C. to 220° C. where spikes in water (μg water detected) and drift (μg/min water) indicate melting and the resulting data can be used to generate melting curves. This KARL FISCHER method was developed to monitor water loss/gain following conditioning of the plastic. The results are shown in FIG. 8. The moisture content in the nucleophilic component dried in accordance with the present invention was about 88% less than the moisture content of the nucelophilic component that was dried by lyophilization. Such reduction in moisture results in a longer shelf-life for the vacuum dried nucelophilic component as compared to the lyophilized nucelophilic component.

### Example 4

### Stability of a Nucelophilic Component During Printing Process

An electrophilic component, that is, an amine-terminated polyethylene glycol (8a15kSS), was melted at 85° C. and held at this temperature in a printer barrel for 24 hours in accordance with one embodiment of the electrophilic printing method of the invention. The stability of the electrophilic component, measured as the N-hydroxysuccinimidyl (NHS) percent substitution, at this temperature was determined as a function of time. The results are shown in FIG. 9. The NHS percent substitution of the 8a15kSS electrophilic component decreased only slightly over the duration of the experiment and remained well above 90% NHS substitution. Thus, the 8a15KSS material has sufficient stability for its deposition by a melt printer.

The preceding description sets forth numerous specific details such as examples of specific systems, components, methods, and so forth, in order to provide a good understanding of several embodiments of the present disclosure. It will be apparent to one skilled in the art, however, that at least some embodiments of the present disclosure may be practiced without these specific details. In other instances, well-known components or methods are not described in detail or are presented in simple block diagram format in order to avoid unnecessarily obscuring the present disclosure. Thus, the specific details set forth are merely exemplary. Particular implementations may vary from these exemplary details and still be contemplated to be within the scope of the present disclosure.

It is to be understood that the above description is intended to be illustrative, and not restrictive. Many other embodiments will be apparent to those of skill in the art upon reading and understanding the above description. The scope of the disclosure should, therefore, be determined with reference to the appended claims, along with the full scope of equivalents to which such claims are entitled.

1. An ocular sealant system, comprising:
a nucleophilic group-containing crosslinker component comprising less than about 500 ppm by weight water;
an electrophilic group-containing multi-arm-polymer precursor component comprising less than about 1 wt. % water; and
a solvent comprising at least one salt.

2. The system of claim 1, further comprising a container configured to store the nucleophilic group-containing crosslinker component and the electrophilic group-containing multi-arm-polymer precursor component.

3. The system of claim 1 or 2, wherein the container comprises one or more of a high-density polyethylene, a polycarbonate, a polystyrene, a nylon or an aluminum foil.

4. The system of any preceding claim, wherein the container comprises less than about 0.50 wt. % water, less than about 0.25 wt. % water, less than about 0.20 wt. % water, or less than about 0.15 wt. % water based on the total weight of the container.

5. The system of any preceding claim, wherein the container comprises a water absorbance value of less than about 0.50% water, less than about 0.25% water, less than about 0.20% water, or less than about 0.15% water.

6. The system of any preceding claim, wherein the container comprises a basin configured to store the nucleophilic group-containing crosslinker component.

7. The system of claim 6, wherein the nucleophilic group-containing crosslinker component is a dried solid.

8. The system of any preceding claim, wherein the container comprises a recess comprising the electrophilic group-containing multi-arm-polymer precursor component in a patterned configuration.

9. The system of any preceding claim, wherein the patterned configuration provides a dissolution of the electrophilic group-containing multi-arm-polymer precursor component into a solution in less than about 5 minutes, less than about 2 minutes, less than about 1 minute, less than about 30 seconds, or less than about 5 seconds.

10. The system of claim 8 or 9, wherein the patterned configuration comprises spaced apart lines of the electrophilic group-containing multi-arm-polymer precursor component.

11. The system of any one of claims 8 to 10, wherein the patterned configuration comprises dots having a diameter of about 1 μm to about 2,000 μm, about 5 μm to about 1,500 μm, about 10 μm to about 1,000 μm, or about 100 μm to about 500 μm.

12. The system of claim 11, wherein each dot comprises about 5 μg to about 30 μg, or about 10 μg to about 17 μg of the electrophilic group-containing multi-arm-polymer precursor component.

13. The system of claim 11 or 12, wherein each dot is spaced apart from an adjacent dot by about 25 μm to about 2000 μm, or about 50 μm to about 1000 μm.

14. The system of any preceding claim, wherein the nucleophilic group-containing crosslinker component comprises a crosslinking agent having one or more of amine functional groups, thiol functional groups, carboxyl functional groups or carbonyl functional groups.

15. The system of any preceding claim, wherein the nucleophilic group-containing crosslinker component comprises a crosslinking agent having a small molecule amine with a molecular weight below about 100,000 Da, below about 50,000 Da, below about 25,000 Da, below about 10,000 Da, or below about 1,000 Da, comprising two or more primary aliphatic amine groups.

16. The system of any preceding claim, wherein the nucleophilic group-containing crosslinker component comprises one or more of dilysine, trilysine, tetralysine, ethylenediamine, 1,3-diaminopropane, 1,3-diaminopropane, diethylenetriamine, trimethylhexamethylenediamine, amine terminated polyethylene glycol, thiol terminated polyethylene glycol, pharmaceutically acceptable salts, hydrates, derivatives and conjugates thereof.

17. The system of any preceding claim, wherein the electrophilic group-containing multi-arm-polymer precursor component comprises polyethylene glycol.

18. The system of any preceding claim, wherein the electrophilic group-containing multi-arm-polymer precursor component comprises one or more of polyethylene glycol units, amine functional groups, thiol functional groups, N-hydroxysuccinimides functional groups or sulfosuccinimides functional groups.

19. The system of any preceding claim, wherein the electrophilic group-containing multi-arm-polymer precursor component comprises one or more of polyethylene glycol, polyethylene oxide, polypropylene oxide, polyvinyl alcohol, poly(vinylpyrrolidinone), polylactic acid, polylactic-co-glycolic acid, or random or block copolymers.

20. The system of any preceding claim, wherein the electrophilic group-containing multi-arm-polymer precursor component is not a lyophilized solid.

21. The system of any preceding claim, wherein the electrophilic group-containing multi-arm-polymer precursor component is derived from a printed melt of polyethylene glycol.

22. The system of any preceding claim, wherein the solvent is configured to dissolve the nucleophilic group-containing crosslinker component and the electrophilic group-containing multi-arm-polymer precursor component to form a solution, and
wherein at least one salt comprises a phosphate salt and a borate salt,
optionally, wherein salt comprises sodium phosphate dibasic, sodium phosphate monobasic and sodium borate decahydrate.

23. The system of any preceding claim, wherein the container comprises a recess comprising a basin configured to store the nucleophilic group-containing crosslinker component.

24. The system of claim 23, wherein the recess is configured to hold the electrophilic group-containing multi-arm-polymer precursor component and the solvent during mixing.

25. The system of any preceding claim, wherein the nucleophilic group-containing crosslinker component comprises about 1.0 wt. % to about 10 wt. % trilysine acetate based on the total weight of the formulation.

26. The system of any preceding claim, wherein the electrophilic group-containing multi-arm-polymer precursor component comprises about 80 wt. % to about 99 wt. % polyethylene glycol based on the total weight of the formulation, optionally, wherein the electrophilic group-containing multi-arm-polymer precursor component comprises a mass of about 5.0 mg to about 20.0 mg or about 8.5 mg to about 15 mg.

27. The system of any preceding claim, wherein the electrophilic group-containing multi-arm-polymer precursor component comprises polyethylene glycol having a molecular weight of about 1000 Da to about 50,000 Da.

28. The system of any preceding claim, wherein the ocular sealant formulation further comprises a solubility enhancer.

29. The system of claim 28, wherein the solubility enhancer comprises about 1 wt. % to about 10 wt. %, about 2 wt. % to about 7 wt. %, or about 3 wt. % to about 5 wt. % D-mannitol.

30. The system of any one of claims 28 to 30, wherein the solubility enhancer comprises one or more of a carrier, an effervescent, a sorbitol isomer, a buffer, a co-solvent, or a surfactant.

31. The system of claim 30, wherein the carrier comprises one or more of mannitol, D-mannitol, sucrose, maltose, lactose, inositol or dextran.

32. The system of claim 30, wherein the sorbitol isomer comprises one or more of mannitol, D-mannitol, xylitol or sorbitol.

33. The system of claim 30, wherein the effervescent comprises one or more of sodium carbonate, sodium bicarbonate, adipic acid, malic acid, tartaric acid, ascorbic acid, fumaric acid, maleic acid, succinic acid or citric acid.

34. The system of claim 30, wherein the buffer comprises one or more of citric acid, ascorbic acid, phosphoric acid, acetic acid, histidine lactic acid, tromethamine gluconic acid, aspartic acid, glutamic acid, tartaric acid, succinic acid, malic acid, fumaric acid, α-ketoglutaric acid, a citrate, a phosphate or an acetate.

35. The system of claim 30, wherein the co-solvent comprises one or more of dimethyl sulfoxide, ethanol, glycerol or benzyl alcohol.

36. The system of claim 30, wherein the surfactant comprises one or more of poly(ethylene glycol) sorbitan monolaurate, a polyethylene glycol-20-sorbitan monolaurate, a polyethylene glycol-80-sorbitan monolaurate), poly(ethylene glycol) ester of castor oil or an ethoxylated 4-tert-octylphenol/formaldehyde condensation polymer.

37. The system of any preceding claim, wherein at least one of the nucleophilic group-containing crosslinker component or the electrophilic group-containing multi-arm-polymer precursor component further comprises an active agent,
optionally wherein the active agent comprises one or more of a cyclodextrin, an antibiotic or a combination thereof,
optionally, wherein the cyclodextrin comprises one or more of acetazolamide, anandamide, a cannabinoid, cyclosporin, a dehydroepiandrosterone, dexamethasone, diclofenac, dipivefrine, fluorometholone, hydrocortisone, loteprednol etabonate or pilocarpine, and
optionally, wherein the antibiotic comprises one or more of azithromycin, bacitracin, neomycin, polymyxin B, mupirocin, nitrofurazone and fusidic acid.

38. The system of any preceding claim, wherein the formulation has a shelf-life of about 9 months to about 4 years.

39. The system of any preceding claim, further comprising an applicator.

40. The system of claim 39, wherein the applicator comprises a thin film of the nucleophilic group-containing crosslinker component.

41. The system of claim 40, wherein the thin film of the nucleophilic group-containing crosslinker component is a dried solid.

42. The system of claim 40 or 41, wherein the thin film further comprises the electrophilic group-containing multi-arm-polymer precursor component.

43. The system of any one of claims 40 to 42, wherein the thin film is configured to dissolve in the solvent in the recess of the container.

44. A method of preparing an ocular sealant formulation system, comprising:
depositing a nucleophilic group-containing crosslinker component on a surface of the container;
drying the nucleophilic group-containing crosslinker component and the container;
depositing a molten electrophilic group-containing multi-arm-polymer precursor component on a surface of the container; and
sealing the container having the nucleophilic group-containing crosslinker component and the electrophilic group-containing multi-arm-polymer precursor component thereon in a storage container.

45. The method of claim 44, further comprising forming the nucleophilic group-containing crosslinker component comprising mixing the nucleophilic group-containing crosslinker component with a colorant to form a mixture.

46. The method of claim 44 or 45, wherein forming the nucleophilic group-containing crosslinker component further comprises adjusting the pH of the mixture.

47. The method of any one of claims 44 to 46, wherein depositing the nucleophilic group-containing crosslinker component comprises dispensing the nucleophilic group-containing crosslinker component in liquid form onto the container.

48. The method of any one of claims 44 to 47, wherein the nucleophilic group-containing crosslinker component is deposited in an amount of about 0.1 μL to about 100 μL, about 0.5 μL to about 50 μL, or about 1 μL to about 25 μL.

49. The method of any one of claims 44 to 48, wherein the container comprises a basin and wherein the nucleophilic group-containing crosslinker component is deposited in the basin.

50. The method of any one of claims 44 to 49, wherein the container comprises one or more of a high-density polyethylene, a polycarbonate, a polystyrene, a nylon or an aluminum foil.

51. The method of any one of claims 44 to 50, further comprising preparing the container for the deposition of the nucleophilic group-containing crosslinker component and the electrophilic group-containing multi-arm-polymer precursor component.

52. The method of any one of claims 44 to 51, further comprising washing the container and drying the container at a temperature of about 30° C. to about 50° C. for about 1 hour to about 24 hours.

53. The method of any one of claims 44 to 52, wherein the container comprises less than about 0.50 wt. % water, less than about 0.25 wt. % water, less than about 0.20 wt. % water, or less than about 0.15 wt. % water based on the total weight of the container.

54. The method of any one of claims 44 to 53, wherein the container comprises a water absorbance value of less than about 0.50% water, less than about 0.25% water, less than about 0.20% water, or less than about 0.15% water.

55. The method of any one of claims 44 to 54, wherein drying the nucleophilic group-containing crosslinker component and the container comprises drying at room temperature or heating under a nitrogen purge at a temperature of about 40° C. to about 80° C.

56. The method of claim 55, comprising heating the nucleophilic group-containing crosslinker component and the container for about 30 minutes to about 4 hours, or about 1 hour to about 2 hours.

57. The method of claim 56, wherein heating further comprises turning off the nitrogen purge and heating the nucleophilic group-containing crosslinker component and the container under a vacuum for about 30 minutes to about 2 hours, or about 1 hour.

58. The method of any one of claims 44 to 57, further comprising cooling the nucleophilic group-containing crosslinker component and the container in an inert environment.

59. The method of any one of claims 44 to 58, wherein depositing the molten electrophilic group-containing multi-arm-polymer precursor component comprises pre-heating a barrel and a nozzle of a printing apparatus to a temperature of about 60° C. to about 200° C.

60. The method of claim 59, wherein depositing the molten electrophilic group-containing multi-arm-polymer precursor component comprises determining that oxygen content and moisture content in the inert environment are less than about 50 ppm by weight, 40 ppm by weight, 30 ppm by weight or 20 ppm by weight.

61. The method of any one of claims 44 to 60, wherein depositing the molten electrophilic group-containing multi-arm-polymer precursor component comprises allowing the electrophilic group-containing multi-arm-polymer precursor component to melt for about 2 min to about 30 min, or about 5 min to about 20 min, or about 10 min to about 15 min.

62. The method of any one of claims 44 to 61, wherein depositing the molten electrophilic group-containing multi-arm-polymer precursor component comprises printing the molten electrophilic group-containing multi-arm-polymer precursor component in a pattern in a recess of the container.

63. The method of claim 62, wherein the pattern comprises one or more of a plurality of spaced apart lines or a plurality of dots.

64. The method of claim 44, wherein the container comprises at least one of a desiccant or an oxygen absorber.

65. The method of claim 64, wherein the at least one of a desiccant or oxygen absorber is comprised in one or more of a liner, a sachet or a canister.

66. The method of any of claims 44 to 65 further comprising, preparing a solvent solution comprising a phosphate salt and a borate salt.

67. The method of claim 66, wherein the solvent comprises sodium phosphate dibasic, sodium phosphate monobasic and sodium borate decahydrate.

68. The method of claim 66 or 67, comprising storing the solvent in a dropper container.

69. The method of any one of claims 44 to 68, wherein the nucleophilic group-containing crosslinker component comprises about 1.0 wt. % to about 10 wt. % trilysine acetate based on the total weight of the formulation.

70. The method of any one of claims 44 to 69, wherein the electrophilic group-containing multi-arm-polymer precursor component comprises about 80 wt. % to about 99 wt. % polyethylene glycol based on the total weight of the formulation, optionally, wherein the electrophilic group-containing multi-arm-polymer precursor component comprises a mass of about 5.0 mg to about 20.0 mg or about 8.5 mg to about 15 mg.

71. The method of any one of claims 44 to 70, wherein the electrophilic group-containing multi-arm-polymer precursor component comprises polyethylene glycol having a molecular weight of about 1000 Da to about 50,000 Da.

72. The method of any one of claims 44 to 70, wherein the nucleophilic group-containing crosslinker component is combined with a solubility enhancer comprising about 1 wt. % to about 10 wt. %, about 2 wt. % to about 7 wt. %, or about 3 wt. % to about 5 wt. % D-mannitol.

73. The method of claim 72, wherein the solubility enhancer comprises one or more of a carrier, an effervescent, a sorbitol isomer, a buffer, a co-solvent, or a surfactant.

74. The method of claim 73, wherein the carrier comprises one or more of mannitol, D-mannitol, sucrose, maltose, lactose, inositol or dextran.

75. The method of claim 73, wherein the sorbitol isomer comprises one or more of mannitol, D-mannitol, xylitol or sorbitol.

76. The method of claim 73, wherein the effervescent comprises one or more of sodium carbonate, sodium bicarbonate, adipic acid, malic acid, tartaric acid, ascorbic acid, fumaric acid, maleic acid, succinic acid or citric acid.

77. The method of claim 73, wherein the buffer comprises one or more of citric acid, ascorbic acid, phosphoric acid, acetic acid, histidine lactic acid, tromethamine gluconic acid, aspartic acid, glutamic acid, tartaric acid, succinic acid, malic acid, fumaric acid, α-ketoglutaric acid, a citrate, a phosphate or an acetate.

78. The method of claim 73, wherein the co-solvent comprises one or more of dimethyl sulfoxide, ethanol, glycerol or benzyl alcohol.

79. The method of claim 73, wherein the surfactant comprises one or more of poly(ethylene glycol) sorbitan monolaurate, a polyethylene glycol-20-sorbitan monolaurate, a polyethylene glycol-80-sorbitan monolaurate), poly(ethylene glycol) ester of castor oil or an ethoxylated 4-tert-octylphenol/formaldehyde condensation polymer.

80. The method of claim 73, wherein at least one of the nucleophilic group-containing crosslinker component or the nucleophilic group-containing multi-arm-polymer precursor component further comprises an active agent,
optionally wherein the active agent comprises one or more of a cyclodextrin, an antibiotic or a combination thereof,
optionally, wherein the cyclodextrin comprises one or more of acetazolamide, anandamide, a cannabinoid, cyclosporin, a dehydroepiandrosterone, dexamethasone, diclofenac, dipivefrine, fluorometholone, hydrocortisone, loteprednol etabonate or pilocarpine, and
optionally, wherein the antibiotic comprises one or more of azithromycin, bacitracin, neomycin, polymyxin B, mupirocin, nitrofurazone and fusidic acid.

81. The method of any one of claims 44 to 80, wherein the formulation has a shelf-life of about 9 months to about 4 years.

82. The method of any one of claims 44 to 81, wherein the nucleophilic group-containing crosslinker component comprises less than about 500 ppm by weight water.

83. The method of any one of claims 44 to 82, a polyethylene glycol comprising less than about 1 wt. % water.

84. An ocular sealant dosage form, comprising:
a compressed mixture of:
a nucleophilic group-containing crosslinker component;
an electrophilic group-containing multi-arm-polymer precursor component; and
at least one excipient,
wherein the ocular sealant dosage form comprises less than about 5 wt. % water.

85. The ocular sealant dosage form of claim 84, wherein the compressed mixture is in the form of one or more of a powder, a granulation, pellets, extrudates or milled particles.

86. The ocular sealant dosage form of claim 84 or 85, wherein the dosage form is a tablet.

87. The ocular sealant dosage form of claim 84, wherein the dosage form is a solid applicator.

88. The ocular sealant dosage form of any of claims 84 to 87, wherein the electrophilic group-containing multi-arm-polymer precursor component is non-lyophilized.

89. The ocular sealant dosage form of claims 84 to 88, comprising about 20 wt. % to about 75 wt. %, about 25 wt. % to about 60 wt. %, or about 37 wt. % to about 55 wt. % of the electrophilic group-containing multi-arm-polymer precursor component based on the total weight of the formulation.

90. The ocular sealant dosage form of any one of claims 84 to 89, wherein the electrophilic group-containing multi-arm-polymer precursor component comprises polyethylene glycol having a molecular weight of about 1000 Da to about 50,000 Da.

91. The ocular sealant dosage form of any one of claims 84 to 90, wherein the compressed mixture further comprises a solubility enhancer.

92. The ocular sealant dosage form of claim 91, wherein the solubility enhancer comprises one or more of a carrier, an effervescent, a sorbitol isomer, a buffer, a co-solvent, or a surfactant.

93. The ocular sealant dosage form of claim 92, wherein the carrier comprises one or more of mannitol, D-mannitol, sucrose, maltose, lactose, sucrose, inositol or dextran.

94. The ocular sealant dosage form of claim 92, wherein the sorbitol isomer comprises one or more of mannitol, D-mannitol, xylitol or sorbitol.

95. The ocular sealant dosage form of claim 92, wherein the effervescent comprises one or more of sodium carbonate, sodium bicarbonate, adipic acid, malic acid, tartaric acid, ascorbic acid, fumaric acid, maleic acid, succinic acid or citric acid.

96. The ocular sealant dosage form of claim 92, wherein the buffer comprises one or more of citric acid, ascorbic acid, phosphoric acid, acetic acid, histidine lactic acid, tromethamine gluconic acid, aspartic acid, glutamic acid, tartaric acid, succinic acid, malic acid, fumaric acid, α-ketoglutaric acid, a citrate, a phosphate or an acetate.

97. The ocular sealant dosage form of claim 92, wherein the co-solvent comprises one or more of dimethyl sulfoxide, ethanol, glycerol or benzyl alcohol.

98. The ocular sealant dosage form of claim 92, wherein the surfactant comprises one or more of poly(ethylene glycol) sorbitan monolaurate, a polyethylene glycol-20-sorbitan monolaurate, a polyethylene glycol-80-sorbitan monolaurate), poly(ethylene glycol) ester of castor oil or an ethoxylated 4-tert-octylphenol/formaldehyde condensation polymer.

99. The ocular sealant dosage form of claim 92, further comprising an active agent,
optionally wherein the active agent comprises one or more of a cyclodextrin, an antibiotic or a combination thereof,
optionally, wherein the cyclodextrin comprises one or more of acetazolamide, anandamide, a cannabinoid, cyclosporin, a dehydroepiandrosterone, dexamethasone, diclofenac, dipivefrine, fluorometholone, hydrocortisone, loteprednol etabonate or pilocarpine, and
optionally, wherein the antibiotic comprises one or more of azithromycin, bacitracin, neomycin, polymyxin B, mupirocin, nitrofurazone and fusidic acid.

100. The ocular sealant dosage form of any one of claims 84 to 99, further comprising a colorant.

101. The ocular sealant dosage form of claim 100, wherein the colorant is selected from the group consisting of Food Drug and Cosmetic (FD&C) Blue No. 1, FD&C Blue No. 2, FD&C Green No. 3, FD&C Red No. 3, FD&C Yellow No. 5, and FD&C Yellow No. 6.

102. The ocular sealant dosage form of any one of claims 98 to 111, comprising a shelf-life of about 9 months to about 4 years.

103. The ocular sealant dosage form of any one of claims 98 to 112, comprising about 30 wt. % to about 70 wt. %, about 40 wt. % to about 60 wt. %, or about 41 wt. % to about 52 wt. % of the polyethylene glycol amine component.

104. The ocular sealant dosage form of any one of claims 98 to 113, wherein dosage form is a compressed tablet, a powder, a granulation, pellets, extruded units or milled particles.

105. The ocular sealant dosage form of any one of claims 98 to 114, wherein the dosage form is a compressed tablet of milled particles having a particle size distribution of less than about 1,000 μm.

106. The ocular sealant dosage form of any one of claims 98 to 115, wherein the dosage form does not comprise trilysine acetate.

107. The ocular sealant dosage form of any one of claims 98 to 116, wherein the dosage form is configured to dissolve in a solvent in less than about 45 seconds, less than about 40 seconds, less than about 35 seconds, or from about 10 seconds to about 60 seconds.

108. The ocular sealant dosage form of any one of claims 98 to 117, wherein the at least one excipient comprises one or more of a solubility enhancer, an effervescent, a binder, a disintegrant or a lubricant.

109. The ocular sealant dosage form of any one of claims 98 to 118, comprising citric acid in an amount of about 15 wt. % to about 30 wt. %, or about 20 wt. % to about 25 wt. % of the total weight of the dosage form.

110. The ocular sealant dosage form of any one of claims 98 to 119, comprising sodium bicarbonate in an amount of about 15 wt. % to about 30 wt. %, or about 20 wt. % to about 27 wt. % of the total weight of the dosage form.

111. An ocular sealant system, comprising:
an ocular sealant dosage form, comprising:
an electrophilic group-containing multi-arm-polymer precursor component;
a polyethylene glycol amine component; and
at least one excipient,
wherein the ocular sealant dosage form comprises less than about 5 wt. % water; and
a solvent comprising at least one salt.

112. The system of claim 121, wherein the solvent comprises a phosphate salt and a borate salt.

113. The system of claim 121 or 122, wherein the solvent comprises sodium phosphate dibasic, sodium phosphate monobasic and sodium borate decahydrate.

114. The system of any one of claims 121 to 123, wherein the electrophilic group-containing multi-arm-polymer precursor component is non-lyophilized.

115. The system of any one of claims 121 to 124, comprising about 20 wt. % to about 75 wt. %, about 25 wt. % to about 60 wt. %, or about 37 wt. % to about 55 wt. % of polyethylene glycol based on the total weight of the formulation.

116. The system of any one of claims 121 to 125, wherein the electrophilic group-containing multi-arm-polymer precursor component comprises polyethylene glycol having a molecular weight of about 1000 Da to about 50,000 Da.

117. The system of any one of claim 111, wherein the ocular sealant dosage form further comprises a solubility enhancer and optionally at least one of a disintegrant, a binder or a lubricant.

118. The system of claim 117, wherein the solubility enhancer comprises one or more of a carrier, an effervescent, a sorbitol isomer, a buffer, a co-solvent, or a surfactant.

119. The system of claim 118, wherein the carrier comprises one or more of mannitol, D-mannitol, sucrose, maltose, lactose, sucrose, inositol or dextran.

120. The system of claim 118, wherein the sorbitol isomer comprises one or more of mannitol, D-mannitol, xylitol or sorbitol.

121. The system of claim 118, wherein the effervescent comprises one or more of sodium carbonate, sodium bicarbonate, adipic acid, malic acid, tartaric acid, ascorbic acid, fumaric acid, maleic acid, succinic acid or citric acid.

122. The system of claim 118, wherein the buffer comprises one or more of citric acid, ascorbic acid, phosphoric acid, acetic acid, histidine lactic acid, tromethamine gluconic acid, aspartic acid, glutamic acid, tartaric acid, succinic acid, malic acid, fumaric acid, α-ketoglutaric acid, a citrate, a phosphate or an acetate.

123. The system of claim 118, wherein the co-solvent comprises one or more of co-solvent comprises one or more of dimethyl sulfoxide, ethanol, glycerol or benzyl alcohol.

124. The system of claim 118, wherein the surfactant comprises one or more of surfactant comprises one or more of poly(ethylene glycol) sorbitan monolaurate, a polyethylene glycol-20-sorbitan monolaurate, a polyethylene glycol-80-sorbitan monolaurate), poly(ethylene glycol) ester of castor oil or an ethoxylated 4-tert-octylphenol/formaldehyde condensation polymer.

125. The system of claim 118, wherein at least one of the nucleophilic group-containing crosslinker component or the electrophilic group-containing multi-arm-polymer precursor component further comprises an active agent,
optionally wherein the active agent comprises one or more of a cyclodextrin, an antibiotic or a combination thereof,
optionally, wherein the cyclodextrin comprises one or more of acetazolamide, anandamide, a cannabinoid, cyclosporin, a dehydroepiandrosterone, dexamethasone, diclofenac, dipivefrine, fluorometholone, hydrocortisone, loteprednol etabonate or pilocarpine, and
optionally, wherein the antibiotic comprises one or more of azithromycin, bacitracin, neomycin, polymyxin B, mupirocin, nitrofurazone and fusidic acid.

126. The system of any one of claims 111 to 125, further comprising a colorant.

127. The system of claim 126, wherein the colorant is selected from the group consisting of Food Drug and Cosmetic (FD&C) Blue No. 1, FD&C Blue No. 2, FD&C Green No. 3, FD&C Red No. 3, FD&C Yellow No. 5, and FD&C Yellow No. 6.

128. The system of any one of claims 111 to 127, comprising a shelf-life of about 9 months to about 4 years.

129. The system of any one of claims 111 to 128, comprising about 30 wt. % to about 70 wt. %, about 40 wt. % to about 60 wt. %, or about 41 wt. % to about 52 wt. % of the polyethylene glycol amine component.

130. The system of any one of claims 111 to 129, wherein dosage form is a compressed tablet, a powder, a granulation, pellets, extruded units or milled particles.

131. The system of any one of claims 111 to 130, wherein the dosage form is a compressed tablet of milled particles having a particle size distribution of less than about 1,000 μm.

132. The system of any one of claims 111 to 131, wherein the dosage form does not comprise trilysine acetate.

133. The system of any one of claims 111 to 132, wherein the dosage form is configured to dissolve in a solvent in less than about 45 seconds, less than about 40 seconds, less than about 35 seconds, or about 10 seconds to about 60 seconds.

134. A method of preparing an ocular sealant dosage form, comprising:
forming particles comprising an electrophilic group-containing multi-arm-polymer precursor component, a nucleophilic group-containing crosslinker component and at least one excipient;
combining the particles to form a combination; and
compressing the combination to form at least one tablet.

135. The method of claim 134, wherein forming particles is performed independently for each of the electrophilic group-containing multi-arm-polymer precursor component, the nucleophilic group-containing crosslinker component, and the at least one excipient.

136. The method of claim 134 or 135, wherein forming particles comprises milling the electrophilic group-containing multi-arm-polymer precursor component, the nucleophilic group-containing crosslinker component and the at least one excipient.

137. The method of claim 137, wherein at least one of: milling is performed in the presence of dry ice or the method is completed within an inert environment.

138. The method of any of claims 134 to 137, wherein the nucleophilic group-containing crosslinker component comprises a crosslinking agent having one or more of amine functional groups, thiol functional groups, carboxyl functional groups or carbonyl functional groups.

139. The method of any of claims 134 to 138, wherein the nucleophilic group-containing crosslinker component comprises a crosslinking agent having a small molecule amine with a molecular weight below about 100,000 Da, comprising two or more primary aliphatic amine groups.

140. The method of any of claims 134 to 139, wherein the nucleophilic group-containing crosslinker component comprises one or more of dilysine, trilysine, tetralysine, ethylenediamine, 1,3-diaminopropane, 1,3-diaminopropane, diethylenetriamine, trimethylhexamethylenediamine, amine terminated polyethylene glycol, thiol terminated polyethylene glycol, pharmaceutically acceptable salts, hydrates, derivatives and conjugates thereof.

141. The method of any of claims 134 to 140, wherein the electrophilic group-containing multi-arm-polymer precursor component comprises polyethylene glycol.

142. The method of any of claims 134 to 141, wherein the electrophilic group-containing multi-arm-polymer precursor component comprises one or more of polyethylene glycol units, amine functional groups, thiol functional groups, N-hydroxysuccinimides functional groups or sulfosuccinimides functional groups.

143. The method of any of claims 134 to 142, wherein the electrophilic group-containing multi-arm-polymer precursor component comprises one or more of polyethylene glycol, polyethylene oxide, polypropylene oxide, polyvinyl alcohol, poly(vinylpyrrolidinone), polylactic acid, polylactic-co-glycolic acid, or random or block copolymers.

144. The method of any of claims 134 to 143, wherein the electrophilic group-containing multi-arm-polymer precursor component is not a lyophilized solid.

145. The method of any of claims 134 to 144, wherein the electrophilic group-containing multi-arm-polymer precursor component is derived from a printed melt of polyethylene glycol.

146. The method of any one of claims 134 to 145, wherein the ocular sealant formulation further comprises a solubility enhancer, and optionally at least one of a disintegrant, a binder or a lubricant.

147. The method of any one of claims 134 to 146, wherein the ocular sealant formulation further comprises a solubility enhancer comprising about 1 wt. % to about 10 wt. %, about 2 wt. % to about 7 wt. %, or about 3 wt. % to about 5 wt. % D-mannitol.

148. The method of any one of claims 134 to 147, wherein the solubility enhancer comprises one or more of a carrier, an effervescent, a sorbitol isomer, a buffer, a co-solvent, or a surfactant.

149. The method of claim 148, wherein the carrier comprises one or more of mannitol, D-mannitol, sucrose, maltose, lactose, inositol or dextran.

150. The method of claim 148, wherein the sorbitol isomer comprises one or more of mannitol, D-mannitol, xylitol or sorbitol.

151. The method of claim 148, wherein the effervescent comprises one or more of sodium carbonate, sodium bicarbonate, adipic acid, malic acid, tartaric acid, ascorbic acid, fumaric acid, maleic acid, succinic acid or citric acid.

152. The method of claim 148, wherein the buffer comprises one or more of citric acid, ascorbic acid, phosphoric acid, acetic acid, histidine lactic acid, tromethamine gluconic acid, aspartic acid, glutamic acid, tartaric acid, succinic acid, malic acid, fumaric acid, α-ketoglutaric acid, a citrate, a phosphate or an acetate.

153. The method of claim 148, wherein the co-solvent comprises one or more of dimethyl sulfoxide, ethanol, glycerol or benzyl alcohol.

154. The method of claim 148, wherein the surfactant comprises one or more of poly(ethylene glycol) sorbitan monolaurate, a polyethylene glycol-20-sorbitan monolaurate, a polyethylene glycol-80-sorbitan monolaurate), poly(ethylene glycol) ester of castor oil or an ethoxylated 4-tert-octylphenol/formaldehyde condensation polymer.

155. The method of claim 148, wherein at least one of the nucleophilic group-containing crosslinker component or the electrophilic group-containing multi-arm-polymer precursor component further comprises an active agent,
optionally wherein the active agent comprises one or more of a cyclodextrin, an antibiotic or a combination thereof,
optionally, wherein the cyclodextrin comprises one or more of acetazolamide, anandamide, a cannabinoid, cyclosporin, a dehydroepiandrosterone, dexamethasone, diclofenac, dipivefrine, fluorometholone, hydrocortisone, loteprednol etabonate or pilocarpine, and
optionally, wherein the antibiotic comprises one or more of azithromycin, bacitracin, neomycin, polymyxin B, mupirocin, nitrofurazone and fusidic acid.

156. The method of any one of claims 134 to 155, further comprising a colorant.

157. The method of claim 156, wherein the colorant is selected from the group consisting of Food Drug and Cosmetic (FD&C) Blue No. 1, FD&C Blue No. 2, FD&C Green No. 3, FD&C Red No. 3, FD&C Yellow No. 5, and FD&C Yellow No. 6.

158. An ocular sealant formulation, comprising:
a polymer network formed by combining:
a nucleophilic group-containing crosslinker component comprising less than about 500 ppm by weight water;
an electrophilic group-containing multi-arm-polymer precursor component comprising less than about 10 wt. % water; and
a solvent comprising at least one salt.

159. The ocular sealant formulation of claim 158, wherein the nucleophilic group-containing crosslinker component comprises a crosslinking agent having one or more of amine functional groups, thiol functional groups, carboxyl functional groups or carbonyl functional groups.

160. The ocular sealant formulation of claim 158 or 159, wherein the nucleophilic group-containing crosslinker component comprises a crosslinking agent having a small molecule amine with a molecular weight below 1,000 Da, comprising two or more primary aliphatic amine groups.

161. The ocular sealant formulation of any one of claims 158 to 160, wherein the nucleophilic group-containing crosslinker component comprises one or more of dilysine, trilysine, tetralysine, ethylenediamine, 1,3-diaminopropane, 1,3-diaminopropane, diethylenetriamine, trimethylhexamethylenediamine, amine terminated polyethylene glycol, thiol terminated polyethylene glycol, pharmaceutically acceptable salts, hydrates, derivatives and conjugates thereof.

162. The ocular sealant formulation of any one of claims 158 to 161, wherein the electrophilic group-containing multi-arm-polymer precursor component comprises polyethylene glycol.

163. The ocular sealant formulation of any one of claims 158 to 162, wherein the electrophilic group-containing multi-arm-polymer precursor component comprises one or more of polyethylene glycol units, amine functional groups, thiol functional groups, N-hydroxysuccinimides functional groups or sulfosuccinimides functional groups.

164. The ocular sealant formulation of any one of claims 158 to 163, wherein the electrophilic group-containing multi-arm-polymer precursor component comprises one or more of polyethylene glycol, polyethylene oxide, polypropylene oxide, polyvinyl alcohol, poly(vinylpyrrolidinone), polylactic acid, polylactic-co-glycolic acid, or random or block copolymers.

165. The ocular sealant formulation of any one of claims 158 to 164, wherein the electrophilic group-containing multi-arm-polymer precursor component is derived from non-lyophilized polyethylene glycol.

166. The ocular sealant formulation of any one of claim 158 or 165, wherein the electrophilic group-containing multi-arm-polymer precursor component is derived from a printed melt of polyethylene glycol.

167. The ocular sealant formulation of any one of claims 158 to 166, wherein the ocular sealant formulation is comprised in a container, the container comprising one or more of high-density polyethylene, polycarbonate, polystyrene or nylon.

168. The ocular sealant formulation of any one of claims 158 to 167, comprising about 1.0 wt. % to about 10 wt. % of trilysine acetate based on the total weight of the formulation.

169. The ocular sealant formulation of any one of claims 158 to 168, comprising about 80 wt. % to about 99 wt. % of polyethylene glycol based on the total weight of the formulation, optionally, wherein the electrophilic group-containing multi-arm-polymer precursor component is in an amount of about 5.0 mg to about 20.0 mg or about 8.5 mg to about 15 mg.

170. The ocular sealant formulation of any one of claims 158 to 169, further comprising a solubility enhancer.

171. The ocular sealant formulation of any one of claims 158 to 170, further comprising a solubility enhancer comprising about 1 wt. % to about 10 wt. %, about 2 wt. % to about 7 wt. %, or about 3 wt. % to about 5 wt. % D-mannitol.

172. The ocular sealant formulation of any one of claims 158 to 171, wherein the electrophilic group-containing multi-arm-polymer precursor component comprises polyethylene glycol having a molecular weight of about 1000 Da to about 50,000 Da.

173. The ocular sealant formulation of any one of claims 158 to 172, wherein the solubility enhancer comprises one or more of a carrier, an effervescent, a sorbitol isomer, a buffer, a co-solvent, or a surfactant.

174. The ocular sealant formulation of claim 173, wherein the carrier comprises one or more of mannitol, D-mannitol, sucrose, maltose, lactose, inositol or dextran.

175. The ocular sealant formulation of claim 173, wherein the sorbitol isomer comprises one or more of mannitol, D-mannitol, xylitol or sorbitol.

176. The ocular sealant formulation of claim 173, wherein the effervescent comprises one or more of sodium carbonate, sodium bicarbonate, adipic acid, malic acid, tartaric acid, ascorbic acid, fumaric acid, maleic acid, succinic acid or citric acid.

177. The ocular sealant formulation of claim 173, wherein the buffer comprises one or more of citric acid, ascorbic acid, phosphoric acid, acetic acid, histidine lactic acid, tromethamine gluconic acid, aspartic acid, glutamic acid, tartaric acid, succinic acid, malic acid, fumaric acid, α-ketoglutaric acid, a citrate, a phosphate or an acetate.

178. The ocular sealant formulation of claim 173, wherein the co-solvent comprises one or more of dimethyl sulfoxide, ethanol, glycerol or benzyl alcohol.

179. The ocular sealant formulation of claim 173, wherein the surfactant comprises one or more of poly(ethylene glycol) sorbitan monolaurate, a polyethylene glycol-20-sorbitan monolaurate, a polyethylene glycol-80-sorbitan monolaurate), poly(ethylene glycol) ester of castor oil or an ethoxylated 4-tert-octylphenol/formaldehyde condensation polymer.

180. The ocular sealant formulation of claim 173, wherein at least one of the nucleophilic group-containing crosslinker component or the electrophilic group-containing multi-arm-polymer precursor component further comprises an active agent,
optionally wherein the active agent comprises one or more of a cyclodextrin, an antibiotic or a combination thereof,
optionally, wherein the cyclodextrin comprises one or more of acetazolamide, anandamide, a cannabinoid, cyclosporin, a dehydroepiandrosterone, dexamethasone, diclofenac, dipivefrine, fluorometholone, hydrocortisone, loteprednol etabonate or pilocarpine, and
optionally, wherein the antibiotic comprises one or more of azithromycin, bacitracin, neomycin, polymyxin B, mupirocin, nitrofurazone and fusidic acid.

181. The ocular sealant formulation of any one of claims 158 to 180, further comprising a colorant.

182. The ocular sealant formulation of claim 181, wherein the colorant is selected from the group consisting of Food Drug and Cosmetic (FD&C) Blue No. 1, FD&C Blue No. 2, FD&C Green No. 3, FD&C Red No. 3, FD&C Yellow No. 5, and FD&C Yellow No. 6.

183. The ocular sealant formulation of any one of claims 158 to 182, comprising a shelf-life of about 9 months to about 4 years.
